(19) United States
(12) Patent Application Publication
(10) Pub. No.: US 2026/0328723 A1
(43) Pub. Date: Oct. 22, 2026

(54) GAS SENSOR

(71) Applicant: NGK INSULATORS, LTD., Nagoya-shi (JP)

(72) Inventor: Ryota TOMIOKA, Nagoya-shi (JP)

(21) Appl. No.: 19/556,127

(22) Filed: Mar. 4, 2026

(30) Foreign Application Priority Data
Mar. 7, 2025 (JP) 2025-036868

(51) Int. Cl. G01N 27/41; G01N 27/409; G01N 33/00

(52) U.S. Cl. CPC G01N 27/41; G01N 27/409; G01N 33/004

(57) ABSTRACT

A gas sensor includes a sensor element and a control unit. The sensor element includes: a measurement-object gas flow cavity having a gas inlet, a first internal cavity that communicates with the gas inlet via a first diffusion-rate limiting path, a second internal cavity that communicates with the first internal cavity via a second diffusion-rate limiting path, and a third internal cavity that communicates with the first internal cavity via a third diffusion-rate limiting path; an oxygen pump cell; a first measurement pump cell; and a second measurement pump cell. The control unit includes a pump control part and a concentration calculating part. The concentration calculating part calculates a water vapor concentration based on a current in the first measurement pump cell, and a carbon dioxide concentration based on the current in the first measurement pump cell and a current in the second measurement pump cell.

### CROSS-REFERENCE TO RELATED APPLICATION

The present application claims priority from Japanese Patent Application No. 2025-036868, filed on Mar. 7, 2025, the content of which is hereby incorporated by reference into this application.

### BACKGROUND OF THE INVENTION

### Technical Field of the Invention

The present invention relates to a gas sensor for detecting water vapor and carbon dioxide in a measurement-object gas.

### Background Art

Measurement of concentration of an objective gas component (oxygen O₂, water vapor H₂O, carbon dioxide CO₂, nitrogen oxide NOx, ammonia NH₃, hydrocarbon HC, etc.) in a measurement-object gas is required in various fields, for example, in combustion control and exhaust gas control of an internal combustion engine such as an engine of automobile, in environment control, in a medical field, a biotechnology field, and an agriculture and industry field. Various measurement devices are used for the concentration measurement, and as an example, a limiting current-type gas sensor using an oxygen ion conductive solid electrolyte such as zirconia (ZrO₂) is known.

For example, JP 5918177 B2 discloses a gas sensor that identifies concentrations of a water vapor component and a carbon dioxide component of a measurement gas based on a current flowing through a solid electrolyte.

### CITATION LIST

### Patent Document

Patent Document 1: JP 5918177 B2

### SUMMARY OF THE INVENTION

### Problems to be Solved by the Invention

As described above, the gas sensor disclosed in JP 5918177 B2 measures each concentration of two gas components, namely, the water vapor component and the carbon dioxide component. In such a gas sensor, it is required to accurately measure both of a water vapor concentration and a carbon dioxide concentration.

It is therefore an object of the present invention to provide a gas sensor that can accurately measure both of a water vapor concentration and a carbon dioxide concentration in a measurement-object gas.

### Means for Solving the Problems

As a result of intensive studies, the present inventor has found that, by decomposing (or, reducing) water vapor and carbon dioxide in a measurement-object gas in a first internal cavity, and distributing the measurement-object gas containing hydrogen and carbon monoxide generated by the decomposition to a second internal cavity and a third internal cavity, it is possible to measure both of a water vapor concentration and a carbon dioxide concentration in the measurement-object gas more accurately. The present invention includes the following aspects.

(1) A gas sensor for detecting water vapor and carbon dioxide in a measurement-object gas, the gas sensor comprising a sensor element and a control unit for controlling the sensor element, wherein
the sensor element comprises:
   a base part in an elongated plate shape, including an oxygen-ion-conductive solid electrolyte layer;
   a measurement-object gas flow cavity having a gas inlet that opens on a surface of the base part, a first internal cavity that communicates with the gas inlet via a first diffusion-rate limiting path, a second internal cavity that communicates with the first internal cavity via a second diffusion-rate limiting path, and a third internal cavity that communicates with the first internal cavity via a third diffusion-rate limiting path;
   an oxygen pump cell including an intracavity oxygen pump electrode disposed in the first internal cavity of the measurement-object gas flow cavity, and an extracavity oxygen pump electrode disposed at a position different from the measurement-object gas flow cavity on the base part and corresponding to the intracavity oxygen pump electrode;
   a first measurement pump cell including an intracavity first measurement electrode disposed in the second internal cavity of the measurement-object gas flow cavity, and an extracavity first measurement electrode disposed at a position different from the measurement-object gas flow cavity on the base part and corresponding to the intracavity first measurement electrode; and
   a second measurement pump cell including an intracavity second measurement electrode disposed in the third internal cavity of the measurement-object gas flow cavity, and an extracavity second measurement electrode disposed at a position different from the measurement-object gas flow cavity on the base part and corresponding to the intracavity second measurement electrode; and
the control unit comprises:
   a pump control part for controlling operation of the oxygen pump cell, the first measurement pump cell and the second measurement pump cell, and
   a concentration calculating part for calculating a concentration of each of water vapor and carbon dioxide in a measurement-object gas, wherein
the pump control part applies a predetermined oxygen pump voltage between the intracavity oxygen pump electrode and the extracavity oxygen pump electrode of the oxygen pump cell to pump out oxygen from the first internal cavity and decompose the water vapor and the carbon dioxide in the measurement-object gas in the first internal cavity;
the pump control part applies a predetermined first measurement pump voltage between the intracavity first measurement electrode and the extracavity first measurement electrode of the first measurement pump cell to pump oxygen into the second internal cavity and oxidize, in the second internal cavity, hydrogen generated by decomposition of the water vapor in the first internal cavity and introduced into the second internal cavity; and
the pump control part applies a predetermined second measurement pump voltage between the intracavity second measurement electrode and the extracavity second measurement electrode of the second measurement pump cell to pump oxygen into the third internal cavity, and oxidize, in the third internal cavity, hydrogen generated by the decomposition of the water vapor in the first internal cavity and introduced into the third internal cavity, and, carbon monoxide generated by decomposition of the carbon dioxide in the first internal cavity and introduced into the third internal cavity; and
the concentration calculating part calculates a water vapor concentration in the measurement-object gas based on a current flowing through the first measurement pump cell; and the concentration calculating part calculates a carbon dioxide concentration in the measurement-object gas based on the current flowing through the first measurement pump cell and a current flowing through the second measurement pump cell.

(2) The gas sensor according to the above (1), wherein the second internal cavity and the third internal cavity are formed independently of each other.

(3) The gas sensor according to the above (1) or (2), wherein the measurement-object gas is distributed at a predetermined ratio and introduced into the second internal cavity from the first internal cavity via the second diffusion-rate limiting path, and into the third internal cavity from the first internal cavity via the third diffusion-rate limiting path, respectively.

(4) The gas sensor according to any one of the above (1) to (3), wherein
the sensor element further comprises:
   a reference gas chamber formed inside the base part, and being separated from the measurement-object gas flow cavity; and
   a reference electrode disposed in the reference gas chamber; and
the pump control part applies the predetermined oxygen pump voltage between the intracavity oxygen pump electrode and the extracavity oxygen pump electrode of the oxygen pump cell based on a voltage between the intracavity oxygen pump electrode and the reference electrode to pump out the oxygen from the first internal cavity and decompose the water vapor and the carbon dioxide in the measurement-object gas in the first internal cavity;
the pump control part applies the predetermined first measurement pump voltage between the intracavity first measurement electrode and the extracavity first measurement electrode of the first measurement pump cell based on a voltage between the intracavity first measurement electrode and the reference electrode to pump the oxygen into the second internal cavity and oxidize, in the second internal cavity, the hydrogen generated by the decomposition of the water vapor in the first internal cavity and introduced into the second internal cavity; and
the pump control part applies the predetermined second measurement pump voltage between the intracavity second measurement electrode and the extracavity second measurement electrode of the second measurement pump cell based on a voltage between the intracavity second measurement electrode and the reference electrode to pump the oxygen into the third internal cavity, and oxidize, in the third internal cavity, the hydrogen generated by the decomposition of the water vapor in the first internal cavity and introduced into the third internal cavity, and the carbon monoxide generated by the decomposition of the carbon dioxide and introduced into the third internal cavity.

(5) The gas sensor according to the above (4), wherein the voltage between the intracavity oxygen pump electrode and the reference electrode is controlled at 300 mV or more and 700 mV or less.

(6) The gas sensor according to any one of the above (1) to (5), wherein a current ratio of the current flowing through the first measurement pump cell to the current flowing through the second measurement pump cell is smaller than 7.25, when the measurement-object gas with the water vapor concentration of 10% and nitrogen as a remainder is introduced into the measurement-object gas flow cavity.

(7) The gas sensor according to any one of the above (1) to (6), wherein a diffusion resistance of the second diffusion-rate limiting path is larger than a diffusion resistance of the third diffusion-rate limiting path.

(8) The gas sensor according to any one of the above (1) to (7), wherein the concentration calculating part calculates the carbon dioxide concentration in the measurement-object gas based on the water vapor concentration calculated based on the current flowing through the first measurement pump cell, and the current flowing through the second measurement pump cell.

(9) The gas sensor according to any one of the above (1) to (8), wherein the concentration calculating part further calculates an oxygen concentration in the measurement-object gas based on a current flowing through the oxygen pump cell, the current flowing through the first measurement pump cell, and the current flowing through the second measurement pump cell.

(10) The gas sensor according to the above (9), wherein the concentration calculating part calculates the oxygen concentration in the measurement-object gas based on the current flowing through the oxygen pump cell, the water vapor concentration calculated based on the current flowing through the first measurement pump cell, and the carbon dioxide concentration calculated based on the current flowing through the first measurement pump cell and the current flowing through the second measurement pump cell.

(11) A control method of a gas sensor for detecting water vapor and carbon dioxide in a measurement-object gas, the gas sensor being the gas sensor according to any one of the above (1) to (10), and
the control method comprising:
   a water vapor concentration measuring step of calculating a water vapor concentration in the measurement-object gas based on a current flowing through the first measurement pump cell; and
   a carbon dioxide concentration measuring step of calculating a carbon dioxide concentration in the measurement-object gas based on the current flowing through the first measurement pump cell and a current flowing through the second measurement pump cell.

### Advantageous Effect of the Invention

According to the present invention, it is possible to provide a gas sensor that can accurately measure both of a water vapor concentration and a carbon dioxide concentration in a measurement-object gas.

### BRIEF DESCRIPTION OF THE DRAWINGS

FIG. 1 is a vertical sectional schematic view in a longitudinal direction of a sensor element 101, showing one example of a schematic configuration of a gas sensor 100.

FIG. 2 is a block diagram showing electric connections between a control unit 90 and respective pump cells 21, 50 and 41, and respective sensor cells 80, 81, 82, and 83 of the sensor element 101, in the gas sensor 100.

FIG. 3 is a vertical sectional schematic view in a longitudinal direction of a sensor element 901, showing one example of a schematic configuration of a gas sensor 900 of a comparative form.

FIG. 4 is a conceptual diagram schematically showing a measurement principle of the gas sensor 900 and the behavior of the measurement-object gas in the respective internal cavities 20, 940, and 961.

FIG. 5 is a schematic diagram showing a relationship between a pump current Ip1 flowing through the first measurement pump cell 50, and a voltage V1 between the first measurement electrode 51 and the reference electrode 42, in the gas sensor 900. The horizontal axis of the FIG. 5 represents the voltage V1 [mV], with the voltage increasing from right to left. The vertical axis of FIG. 5 represents the pump current Ip1 [μA].

FIG. 6 is a conceptual diagram schematically showing a measurement principle of the gas sensor 100 and the behavior of the measurement-object gas in the respective internal cavities 20, 40, and 61.

FIG. 7 is a schematic diagram showing a relationship between the pump current Ip1 flowing through the first measurement pump cell 50, and the voltage V1 between the first measurement electrode 51 and the reference electrode 42, and shows comparison between the gas sensor 100 of the embodiment according to the present invention and the gas sensor 900 of the example of the conventional gas sensor. The horizontal axis of the FIG. 7 represents the voltage V1 [mV], with the voltage increasing from right to left. The vertical axis of FIG. 7 represents the pump current Ip1 [μA].

FIGS. 8A to 8C show schematic diagrams for explanation of a method for calculating a water vapor concentration, a carbon dioxide concentration, and an oxygen concentration by using pump currents Ip1, Ip2 and Ip0. FIG. 8A is a schematic diagram for calculating the water vapor concentration (H₂O concentration). The horizontal axis of the diagram represents the H₂O concentration [%] and the vertical axis of the diagram represents the pump current Ip1 [μA]. FIG. 8B is a schematic diagram for calculating the carbon dioxide concentration (CO₂ concentration). The horizontal axis of the diagram represents the H₂O concentration [%] and the vertical axis of the diagram represents the pump current Ip2 [μA]. FIG. 8C is a schematic diagram for calculating the oxygen concentration (O₂ concentration). The horizontal axis of the diagram represents the H₂O concentration [%] and the vertical axis of the diagram represents the pump current Ip0 [μA].

FIG. 9 is a vertical sectional schematic view in a longitudinal direction of the sensor element 201, showing one example of a general configuration of a gas sensor 200 including the sensor element 201.

FIG. 10 is a partial sectional schematic view of the sensor element 201, showing a part of the section along line X-X in FIG. 9. FIG. 10 is a schematic view showing a general planar arrangement of the inner main pump electrode 22, the first measurement electrode 51, and the second measurement electrode 44.

FIG. 11 is a vertical sectional schematic view in the longitudinal direction of the sensor element 201 along line XI-XI in FIG. 10, showing one example of the general configuration of the gas sensor 200 including the sensor element 201.

FIG. 12 is a conceptual diagram schematically showing a measurement principle of a gas sensor and the behavior of the measurement-object gas in the respective internal cavities 20, 40, and 61.

FIG. 13 is a graph showing a current voltage curve of the pump current Ip1 for H₂O in each of Examples 1 to 5 and Comparative Example 1. The horizontal axis of the graph represents the voltage V1 [mV] and the vertical axis of the graph represents a relative value (Ip1/Ip1_V1=300 mV) [%] of the pump current Ip1 when the pump current Ip1 at the voltage V1 of 300 mV is set to 100%.

FIG. 14 is a graph showing a relationship of the voltage V1 between the first measurement electrode 51 and the reference electrode 44 with the pump current Ip2 in the second measurement pump cell 41 in each of Examples 1 to 5 and Comparative Example 1. The horizontal axis of the graph represents the voltage V1 [mV] and the vertical axis of the graph represents a relative value (Ip2/Ip2_V1=800 mV) [%] of the pump current Ip2 when the pump current Ip2 at the voltage V1 of 800 mV is set to 100%.

### MODES FOR CARRYING OUT OF THE INVENTION

A gas sensor of the present invention is a gas sensor that detects water vapor and carbon dioxide in a measurement-object gas, and includes a sensor element and a control unit for controlling the sensor element.

The sensor element contained in the gas sensor of the present invention includes:
a base part in an elongated plate shape, including an oxygen-ion-conductive solid electrolyte layer;
a measurement-object gas flow cavity having a gas inlet that opens on a surface of the base part, a first internal cavity that communicates with the gas inlet via a first diffusion-rate limiting path, a second internal cavity that communicates with the first internal cavity via a second diffusion-rate limiting path, and a third internal cavity that communicates with the first internal cavity via a third diffusion-rate limiting path;
an oxygen pump cell including an intracavity oxygen pump electrode disposed in the first internal cavity of the measurement-object gas flow cavity, and an extracavity oxygen pump electrode disposed at a position different from the measurement-object gas flow cavity on the base part and corresponding to the intracavity oxygen pump electrode;
a first measurement pump cell including an intracavity first measurement electrode disposed in the second internal cavity of the measurement-object gas flow cavity, and an extracavity first measurement electrode disposed at a position different from the measurement-object gas flow cavity on the base part and corresponding to the intracavity first measurement electrode; and
a second measurement pump cell including an intracavity second measurement electrode disposed in the third internal cavity of the measurement-object gas flow cavity, and an extracavity second measurement electrode disposed at a position different from the measurement-object gas flow cavity on the base part and corresponding to the intracavity second measurement electrode.

The control unit contained in the gas sensor of the present invention includes:
a pump control part for controlling operation of the oxygen pump cell, the first measurement pump cell and the second measurement pump cell, and
a concentration calculating part for calculating a concentration of each of water vapor and carbon dioxide in a measurement-object gas, wherein
the pump control part applies a predetermined oxygen pump voltage between the intracavity oxygen pump electrode and the extracavity oxygen pump electrode of the oxygen pump cell to pump out oxygen from the first internal cavity and decompose the water vapor and the carbon dioxide in the measurement-object gas in the first internal cavity;
the pump control part applies a predetermined first measurement pump voltage between the intracavity first measurement electrode and the extracavity first measurement electrode of the first measurement pump cell to pump oxygen into the second internal cavity and oxidize, in the second internal cavity, hydrogen generated by decomposition of the water vapor in the first internal cavity and introduced into the second internal cavity; and
the pump control part applies a predetermined second measurement pump voltage between the intracavity second measurement electrode and the extracavity second measurement electrode of the second measurement pump cell to pump oxygen into the third internal cavity, and oxidize, in the third internal cavity, hydrogen generated by the decomposition of the water vapor in the first internal cavity and introduced into the third internal cavity, and, carbon monoxide generated by decomposition of the carbon dioxide in the first internal cavity and introduced into the third internal cavity; and
the concentration calculating part calculates a water vapor concentration in the measurement-object gas based on a current flowing through the first measurement pump cell; and
the concentration calculating part calculates a carbon dioxide concentration in the measurement-object gas based on the current flowing through the first measurement pump cell and a current flowing through the second measurement pump cell.

### [Schematic Configuration of Gas Sensor]

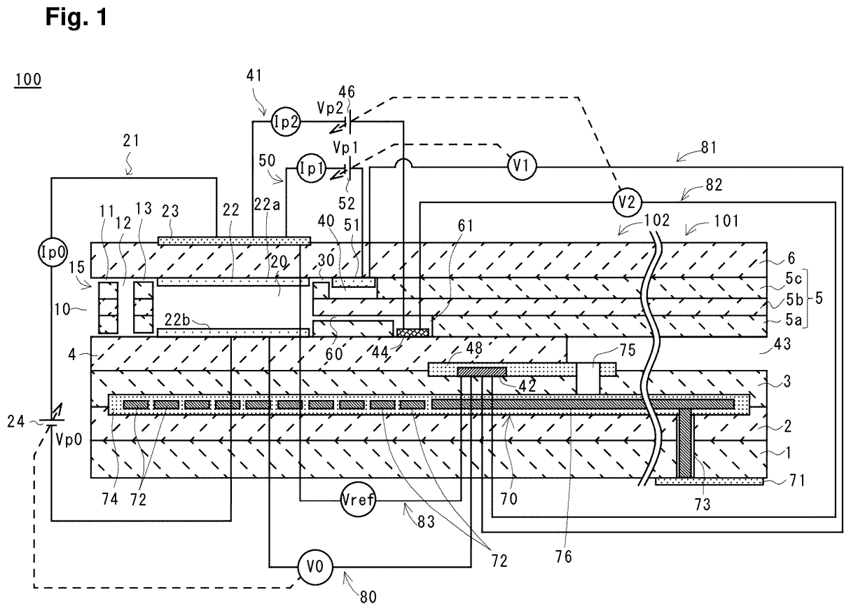

The sensor element of the present invention will now be described with reference to the drawings. FIG. 1 is a vertical sectional schematic view in a longitudinal direction of a sensor element 101, showing one example of a schematic configuration of a gas sensor 100 including the sensor element 101. Hereinafter, based on FIG. 1, the upper side and the lower side in FIG. 1 are respectively defined as top and bottom, and the left side and the right side in FIG. 1 are respectively defined as a front end side and a rear end side.

In the embodiment of FIG. 1, the gas sensor 100 represents one example of a limiting current-type gas sensor that detects water vapor and carbon dioxide in a measurement-object gas by the sensor element 101, and measures the concentrations of the water vapor and the carbon dioxide.

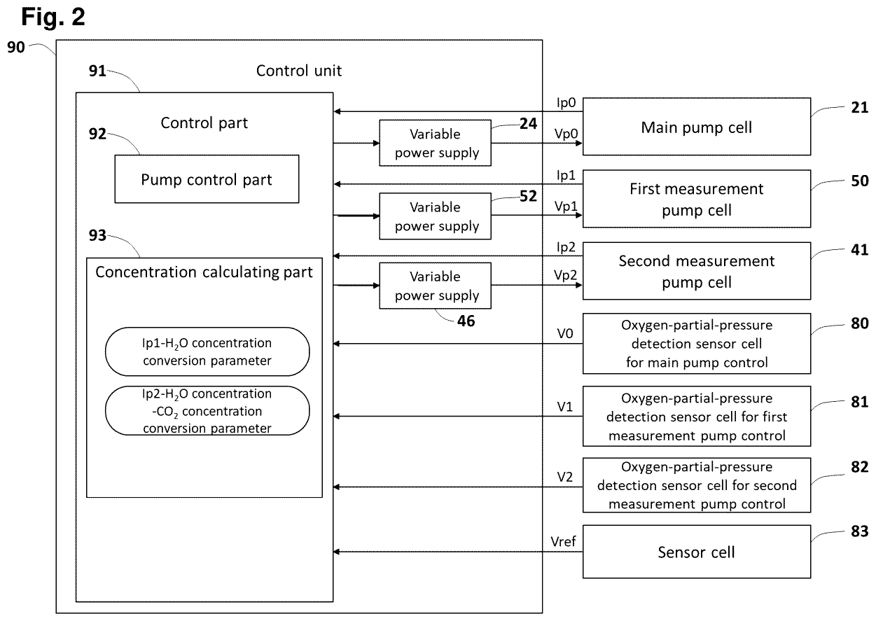

Further, the gas sensor 100 includes a control unit 90 for controlling the sensor element 101. FIG. 2 is a block diagram showing electric connections between the control unit 90 and the sensor element 101.

### (Sensor Element)

The sensor element 101 is an element in an elongated plate shape, including a base part 102 having such a structure that a plurality of oxygen-ion-conductive solid electrolyte layers are layered. The elongated plate shape also called a long plate shape or a belt shape. The base part 102 has such a structure that six layers, namely, a first substrate layer 1, a second substrate layer 2, a third substrate layer 3, a first solid electrolyte layer 4, a spacer layer 5 (5a, 5b, and 5c), and a second solid electrolyte layer 6, are layered in this order from the bottom side, as viewed in the drawing. Each of the six layers is formed of an oxygen-ion-conductive solid electrolyte layer containing, for example, zirconia (ZrO₂). The solid electrolyte forming these six layers is dense and gastight. These six layers all may have the same thickness, or the thickness may vary among the layers. In the sensor element 100, the spacer layer 5 has a three-layered structure that three layers 5a, 5b, and 5c are further layered. The layers are adhered to each other with an adhesive layer of a solid electrolyte interposed therebetween, and the base part 102 includes the adhesive layer. While a layer configuration composed of the six layers is illustrated in FIG. 1, the layer configuration in the present invention is not limited to this, and any number of layers and any layer configuration are possible.

A measurement-object gas flow cavity 15 has a gas inlet 10 that opens on a surface of the base part 102, a first internal cavity 20 that communicates with the gas inlet 10 via a first diffusion-rate limiting path 11 (namely, a first diffusion-rate limiting part), a second internal cavity 40 that communicates with the first internal cavity 20 via a second diffusion-rate limiting path 30 (namely, a second diffusion-rate limiting part), and a third internal cavity 61 that communicates with the first internal cavity 20 via a third diffusion-rate limiting path 60 (namely, a third diffusion-rate limiting part). That is, in this embodiment, the second internal cavity 40 and the third internal cavity 61 are connected with the first internal cavity 20 in parallel. Thus, the measurement-object gas flow cavity 15 is configured so that the measurement-object gas introduced through the gas inlet 10 first reaches the first internal cavity 20, and then is distributed to the second internal cavity 40 and the third internal cavity 61.

The gas inlet 10 is formed between the lower surface of the second solid electrolyte layer 6 and the upper surface of the first solid electrolyte layer 4 in one end part in the longitudinal direction (hereinafter, referred to as a front end part) of the sensor element 101. The measurement-object gas flow cavity 15, that is, a measurement-object gas flow part is formed in such a form that the first diffusion-rate limiting path 11, a buffer space 12, an auxiliary diffusion-rate limiting path 13 (namely, an auxiliary diffusion-rate limiting part), and the first internal cavity 20 communicate in this order in the longitudinal direction from the gas inlet 10, and further, rearward of the first internal cavity 20, the second diffusion-rate limiting path 30 and the second internal cavity 40 communicate in this order, and in parallel with these, the third diffusion-rate limiting path 60 and the third internal cavity 61 communicate in this order.

The gas inlet 10, the buffer space 12, and the first internal cavity 20 constitute internal spaces of the sensor element 101. Each of the internal spaces is provided in such a manner that a portion of the spacer layer 5 (5a, 5b, and 5c) is hollowed out, and the top of each of the internal spaces is defined by the lower surface of the second solid electrolyte layer 6, the bottom of each of the internal spaces is defined by the upper surface of the first solid electrolyte layer 4, and the lateral surface of each of the internal spaces is defined by the lateral surface of the spacer layer 5 (5a, 5b, and 5c).

The second internal cavity 40 constitutes an internal space of the sensor element 101. The internal space is provided in such a manner that a portion of the spacer layer 5c is hollowed out, and the top of the internal space is defined by the lower surface of the second solid electrolyte layer 6, the bottom of the internal space is defined by the upper surface of the spacer layer 5b, and the lateral surface of the internal space is defined by the lateral surface of the spacer layer 5c. The third internal cavity 61 constitutes an internal space of the sensor element 101. The internal space is provided in such a manner that a portion of the spacer layer 5a is hollowed out, and the top of the internal space is defined by the lower surface of the spacer layer 5b, the bottom of the internal space is defined by the upper surface of the first solid electrolyte layer 4, and the lateral surface of the internal space is defined by the lateral surface of the spacer layer 5a.

Each of the first diffusion-rate limiting path 11 and the auxiliary diffusion-rate limiting path 13 is provided as two laterally elongated slits (having the longitudinal direction of the openings in the direction perpendicular to the figure in FIG. 1). Each of the first diffusion-rate limiting path 11 and the auxiliary diffusion-rate limiting path 13 may be in such a form that a desired diffusion resistance is created, but the form is not limited to the slits.

The second diffusion-rate limiting path 30 is provided as a single laterally elongated slit (having the longitudinal direction of the opening in the direction perpendicular to the figure in FIG. 1) between the spacer layer 5c and the second solid electrolyte layer 6. The second diffusion-rate limiting path 30 may be in such a form that a desired diffusion resistance is created, but the form is not limited to the slit.

The third diffusion-rate limiting path 60 is provided as a single laterally elongated slit (having the longitudinal direction of the opening in the direction perpendicular to the figure in FIG. 1) between the spacer layer 5a and the spacer layer 5b. The third diffusion-rate limiting path 60 may be in such a form that a desired diffusion resistance is created, but the form is not limited to the slit.

Also, at a position farther from the front end than the measurement-object gas flow cavity 15, a reference gas introduction space 43 is disposed between the upper surface of the third substrate layer 3 and the lower surface of the spacer layer 5a at a position where the reference gas introduction space 43 is laterally defined by the lateral surface of the first solid electrolyte layer 4. The reference gas introduction space 43 has an opening in the other end part (hereinafter, referred to as a rear end part) of the sensor element 101. As a reference gas for concentration measurement, for example, air is introduced into the reference gas introduction space 43.

An air introduction layer 48 is a layer formed of porous alumina, and is so configured that a reference gas is introduced into the air introduction layer 48 via the reference gas introduction space 43. The air introduction layer 48 is formed to cover a reference electrode 42. In this embodiment, the air introduction layer 48 and the reference gas introduction space 43 correspond to the reference gas chamber of the present invention.

The reference electrode 42 is an electrode disposed in the reference gas chamber. The reference electrode 42 is an electrode sandwiched between the upper surface of the third substrate layer 3 and the first solid electrolyte layer 4, and as described above, the air introduction layer 48 leading to the reference gas introduction space 43 is disposed around the reference electrode 42. That is, the reference electrode 42 is disposed to be in contact with a reference gas via the air introduction layer 48 which is a porous material, and the reference gas introduction space 43. As will be described later, the reference electrode 42 can be used to measure the oxygen concentration (oxygen partial pressure) in the first internal cavity 20, the second internal cavity 40, and the third internal cavity 61. The reference electrode 42 may be formed as a porous cermet electrode (e.g., a cermet electrode of Pt and ZrO₂) in a rectangular shape in a planar view.

In the measurement-object gas flow cavity 15, the gas inlet 10 is open to the external space, and the measurement-object gas is taken into the sensor element 101 from the external space through the gas inlet 10.

In the present embodiment, the measurement-object gas flow cavity 15 is in such a form that the measurement-object gas is introduced through the gas inlet 10 that is open on the front end surface of the sensor element 101, however, the present invention is not limited to this form. For example, the measurement-object gas flow cavity 15 need not have a recess of the gas inlet 10. In this case, the first diffusion-rate limiting path 11 serves as both a gas inlet and a first diffusion-rate limiting path.

For example, the measurement-object gas flow cavity 15 may have an opening that communicates with the buffer space 12 or a position near the buffer space 12 of the first internal cavity 20, on a lateral surface along the longitudinal direction of the base part 102. In this case, the measurement-object gas is introduced from the lateral surface along the longitudinal direction of the base part 102 through the opening.

Further, for example, the measurement-object gas flow cavity 15 may be so configured that the measurement-object gas is introduced through a porous body.

The first diffusion-rate limiting path 11 creates a predetermined diffusion resistance to the measurement-object gas taken through the gas inlet 10.

The buffer space 12 is provided to mitigate the influence of pressure fluctuation on the detected value when the pressure of the measurement-object gas fluctuates. The sensor element 101 may have a structure without the buffer space 12.

The auxiliary diffusion-rate limiting path 13 creates a predetermined diffusion resistance to the measurement-object gas introduced into the first internal cavity 20 from the buffer space 12. The auxiliary diffusion-rate limiting path 13 is provided in association with the buffer space 12 being provided.

When the buffer space 12 and the auxiliary diffusion-rate limiting path 13 are not provided, the first diffusion-rate limiting path 11 directly communicates with the first internal cavity 20.

The first internal cavity 20 is provided as a space for adjusting the oxygen partial pressure in the measurement-object gas introduced through the auxiliary diffusion-rate limiting path 13. The oxygen partial pressure is adjusted by operation of a main pump cell 21. That is, the main pump cell 21 functions as an oxygen pump cell of the present invention.

The main pump cell 21 is an electrochemical pump cell including the inner main pump electrode 22 as an intra-cavity oxygen pump electrode disposed in the first internal cavity 20 of the measurement-object gas flow cavity 15, and an outer pump electrode 23 as an extra-cavity oxygen pump electrode disposed at a position different from the measurement-object gas flow cavity 15 on the base part 102 (in FIG. 1, on an outer surface of the base part 102) and corresponding to the inner main pump electrode 22. The phrase “corresponding to the inner main pump electrode 22” means that the outer pump electrode 23 and the inner main pump electrode 22 are provided with the second solid electrolyte layer 6 being interposed therebetween.

That is, the main pump cell 21 is an electrochemical pump cell composed of the inner main pump electrode 22 having a ceiling electrode portion 22a disposed over substantially the entire surface of the lower surface of the second solid electrolyte layer 6 that faces the first internal cavity 20, the outer pump electrode 23 disposed on the upper surface of the second solid electrolyte layer 6 so as to be exposed to the external space, and the second solid electrolyte layer 6 sandwiched between the inner main pump electrode 22 and the outer pump electrode 23.

The inner main pump electrode 22 is formed to span the upper and lower solid electrolyte layers (the second solid electrolyte layer 6 and the first solid electrolyte layer 4) that define the first internal cavity 20 and the spacer layer 5 (5a, 5b, and 5c) that defines the lateral wall. Specifically, the ceiling electrode portion 22a is formed on the lower surface of the second solid electrolyte layer 6 that defines the ceiling surface of the first internal cavity 20, and a bottom electrode portion 22b is formed on the upper surface of the first solid electrolyte layer 4 that defines the bottom surface of the first internal cavity 20. Also, lateral electrode portions (not shown) are formed on the lateral wall surfaces (inner surface) of the spacer layer 5 (5a, 5b, and 5c) that form both lateral wall parts of the first internal cavity 20 so as to connect the ceiling electrode portion 22a and the bottom electrode portion 22b. Thus, the inner main pump electrode 22 is provided as a tunnel-like structure in the area where the lateral electrode portions are disposed.

The inner main pump electrode 22 and the outer pump electrode 23 are porous cermet electrodes (electrodes in a state that a metal component and a ceramic component are mixed) in a rectangular shape in a planar view. The inner main pump electrode 22 and the outer pump electrode 23 may contain a noble metal having catalytic activity (e.g., at least one of Pt, Rh, Ir, Ru, and Pd) as the metal component. The ceramic component to be used is not particularly limited, but is preferably an oxygen-ion-conductive solid electrolyte as in the case of the base part 102. For example, each of the inner main pump electrode 22 and the outer pump electrode 23 may be a porous cermet electrode made of Pt and ZrO₂.

In the main pump cell 21, a pump voltage (oxygen pump voltage) Vp0 is applied between the inner main pump electrode 22 and the outer pump electrode 23 by a variable power supply 24 to make a pump current Ip0 flow between the inner main pump electrode 22 and the outer pump electrode 23 in either a positive or negative direction, and thus it is possible to pump out oxygen in the first internal cavity 20 to the external space or pump oxygen into the first internal cavity 20 from the external space. In FIG. 1, a direction of pumping out oxygen from the first internal cavity 20 is illustrated as a positive direction.

The inner main pump electrode 22, the second solid electrolyte layer 6, the spacer layer 5 (5a, 5b, and 5c), the first solid electrolyte layer 4, the third substrate layer 3, and the reference electrode 42 form an electrochemical sensor cell, namely, an oxygen-partial-pressure detection sensor cell 80 for main pump control. In the oxygen-partial-pressure detection sensor cell 80 for main pump control, an electromotive force (a voltage V0) is generated between the inner main pump electrode 22 and the reference electrode 42 due to a difference in oxygen concentration between an atmosphere in the first internal cavity 20 and a reference gas in the reference gas introduction space 43.

The oxygen concentration (oxygen partial pressure) in the first internal cavity 20 can be detected from the voltage V0 measured in the oxygen-partial-pressure detection sensor cell 80 for main pump control. In addition, the pump current Ip0 is controlled by performing feedback control of the pump voltage Vp0 in the variable power supply 24 so that the voltage V0 is constant. Thus, the oxygen partial pressure in the first internal cavity 20 can be maintained at a predetermined value.

The second diffusion-rate limiting path 30 creates a predetermined diffusion resistance to the measurement-object gas whose oxygen concentration (oxygen partial pressure) has been controlled in the first internal cavity 20 by the operation of the main pump cell 21, and guides the measurement-object gas into the second internal cavity 40. The third diffusion-rate limiting path 60 creates a predetermined diffusion resistance to the measurement-object gas whose oxygen concentration (oxygen partial pressure) has been controlled in the first internal cavity 20 by the operation of the main pump cell 21, and guides the measurement-object gas into the third internal cavity 61. The measurement-object gas is distributed to the second internal cavity 40 and the third internal cavity 61 in accordance with values of the diffusion resistance in the second diffusion-rate limiting path 30 and the diffusion resistance in the third diffusion-rate limiting path 60.

The second internal cavity 40 is provided as a space for performing processing related to measurement of a water vapor concentration in the measurement-object gas introduced through the second diffusion-rate limiting path 30. By operation of a first measurement pump cell 50, oxygen is supplied from the outside of the sensor element 101 to the second internal cavity 40.

The first measurement pump cell 50 is an electrochemical pump cell including a first measurement electrode 51 as an intracavity first measurement electrode disposed in the second internal cavity 40 of the measurement-object gas flow cavity 15, and an extracavity first measurement electrode disposed at a position different from the measurement-object gas flow cavity 15 on the base part 102 and corresponding to the first measurement electrode 51. In this embodiment, the outer pump electrode 23 disposed on the outer surface of the base part 102 functions also as the extracavity first measurement electrode. The phrase “corresponding to the first measurement electrode 51” means that the outer pump electrode 23 and the first measurement electrode 51 are provided with the second solid electrolyte layer 6 being interposed therebetween.

That is, the first measurement pump cell 50 is an electrochemical pump cell composed of the first measurement electrode 51 disposed on substantially the entire surface of the lower surface of the second solid electrolyte layer 6 facing with the second internal cavity 40, the outer pump electrode 23 (the outer electrode is not limited to the outer pump electrode 23, but may be any suitable electrode disposed at a position different from the measurement-object gas flow cavity 15, for example, outside the sensor element 101), and the second solid electrolyte layer 6.

The first measurement electrode 51 is a porous cermet electrode in a rectangular shape in a planar view, as with the case of the inner main pump electrode 22 and the outer pump electrode 23. The first measurement electrode 51 may contain a noble metal having catalytic activity (e.g., at least one of Pt, Rh, Ir, Ru, and Pd) as the metal component. The ceramic component to be used is not particularly limited, but is preferably an oxygen-ion-conductive solid electrolyte as in the case of the base part 102. For example, the first measurement electrode 51 may be a porous cermet electrode made of Pt containing 0.1 wt. % to 30.0 wt. % of Au and ZrO₂.

In the first measurement pump cell 50, by applying a pump voltage (first measurement pump voltage) Vp1 between the first measurement electrode 51 and the outer pump electrode 23 by a variable power supply 52 to make a pump current Ip1 flow between the first measurement electrode 51 and the outer pump electrode 23, it is possible to pump oxygen into the second internal cavity 40. In FIG. 1, regarding the pump current Ip1, a direction of pumping oxygen into the second internal cavity 40 is illustrated as a positive direction unlike the case of the pump current Ip0.

The first measurement electrode 51, the reference electrode 42, the second solid electrolyte layer 6, the spacer layer 5 (5a, 5b, and 5c), the first solid electrolyte layer 4, and the third substrate layer 3 form an electrochemical sensor cell, namely, an oxygen-partial-pressure detection sensor cell 81 for first measurement pump control. In the oxygen-partial-pressure detection sensor cell 81 for first measurement pump control, an electromotive force (a voltage V1) is generated between the first measurement electrode 51 and the reference electrode 42 due to a difference in oxygen concentration between an atmosphere in the second internal cavity 40 and the reference gas in the reference gas introduction space 43.

The oxygen concentration (oxygen partial pressure) in the second internal cavity 40 can be detected from the voltage V1 measured in the oxygen-partial-pressure detection sensor cell 81 for first measurement pump control. The pump voltage Vp1 in the variable power supply 52 is feedback controlled on the basis of the voltage V1 detected by the oxygen-partial-pressure detection sensor cell 81 for first measurement pump control.

The third internal cavity 61 is provided as a space for performing processing related to measurement of a carbon dioxide concentration in the measurement-object gas introduced through the third diffusion-rate limiting path 60. By operation of a second measurement pump cell 41, oxygen is supplied from the outside of the sensor element 101 to the third internal cavity 61.

The second measurement pump cell 41 is an electrochemical pump cell including a second measurement electrode 44 as an intracavity second measurement electrode disposed in the third internal cavity 61 of the measurement-object gas flow cavity 15, and an extracavity second measurement electrode disposed at a position different from the measurement-object gas flow cavity 15 on the base part 102 and corresponding to the second measurement electrode 44. In this embodiment, the outer pump electrode 23 disposed on the outer surface of the base part 102 functions also as the extracavity second measurement electrode. The phrase “corresponding to the second measurement electrode 44” means that the outer pump electrode 23 and the second measurement electrode 44 are provided with the second solid electrolyte layer 6, the spacer layer 5 (5a, 5b, and 5c), and the first solid electrolyte layer 4 being interposed therebetween.

That is, the second measurement pump cell 41 is an electrochemical pump cell composed of the second measurement electrode 44 disposed on an upper surface of the first solid electrolyte layer 4 facing with the third internal cavity 61, the outer pump electrode 23 (the outer electrode is not limited to the outer pump electrode 23, but may be any suitable electrode disposed at a position different from the measurement-object gas flow cavity 15, for example, outside the sensor element 101), the second solid electrolyte layer 6, the spacer layer 5 (5a, 5b, and 5c), and the first solid electrolyte layer 4.

The second measurement electrode 44 is a porous cermet electrode in a rectangular shape in a planar view, as with the case of the inner main pump electrode 22 and the outer pump electrode 23. The second measurement electrode 44 may contain a noble metal having catalytic activity (e.g., at least one of Pt, Rh, Ir, Ru, and Pd) as the metal component. The ceramic component to be used is not particularly limited, but is preferably an oxygen-ion-conductive solid electrolyte as in the case of the base part 102. For example, the second measurement electrode 44 may be a porous cermet electrode made of Pt and ZrO₂.

In the second measurement pump cell 41, by applying a pump voltage (second measurement pump voltage) Vp2 between the second measurement electrode 44 and the outer pump electrode 23 by a variable power supply 46 to make a pump current Ip2 flow between the second measurement electrode 44 and the outer pump electrode 23, it is possible to pump oxygen into the third internal cavity 61. In FIG. 1, regarding the pump current Ip2, a direction of pumping oxygen into the third internal cavity 61 is illustrated as a positive direction unlike the case of the pump current Ip0.

To detect the oxygen partial pressure around the second measurement electrode 44, the first solid electrolyte layer 4, the third substrate layer 3, the second measurement electrode 44, and the reference electrode 42 constitute an electrochemical sensor cell, namely an oxygen-partial-pressure detection sensor cell 82 for second measurement pump control. In the oxygen-partial-pressure detection sensor cell 82 for second measurement pump control, an electromotive force (a voltage V2) is generated between the second measurement electrode 44 and the reference electrode 42 due to a difference in oxygen concentration between an atmosphere in the third internal cavity 61 and the reference gas in the reference gas introduction space 43.

The oxygen concentration (oxygen partial pressure) in the third internal cavity 61 can be detected from the voltage V2 measured in the oxygen-partial-pressure detection sensor cell 82 for second measurement pump control. The pump voltage Vp2 in the variable power supply 46 is feedback controlled on the basis of the voltage V2 detected by the oxygen-partial-pressure detection sensor cell 82 for second measurement pump control.

Also, the second solid electrolyte layer 6, the spacer layer 5 (5a, 5b, and 5c), the first solid electrolyte layer 4, the third substrate layer 3, the outer pump electrode 23, and the reference electrode 42 constitute an electrochemical sensor cell 83, and it is possible to detect the oxygen partial pressure in the measurement-object gas outside the sensor by an electromotive force (a voltage Vref) obtained by the sensor cell 83. Thus, a function as an oxygen concentration cell may be added to the gas sensor 100.

The sensor element 101 further includes a heater part 70 that functions as a temperature regulator of heating and maintaining the temperature of the sensor element 101 so as to enhance the oxygen ion conductivity of the solid electrolyte. The heater part 70 includes a heater electrode 71, a heater 72, a heater lead 76, a through hole 73, a heater insulating layer 74, and a pressure relief vent 75.

The heater electrode 71 is an electrode formed in contact with the lower surface of the first substrate layer 1. The power can be supplied to the heater part 70 from the outside by connecting the heater electrode 71 with an external power supply.

The heater 72 is an electrical resistor sandwiched by the second substrate layer 2 and the third substrate layer 3 from top and bottom. The heater 72 is connected with the heater electrode 71 via the heater lead 76 that connects with the heater 72 and extends in the rear end side in the longitudinal direction of the sensor element 101, and the through hole 73. The heater 72 is externally powered through the heater electrode 71 to generate heat, and heats and maintains the temperature of the solid electrolyte forming the sensor element 101.

The heater 72 is embedded over the whole area from the first internal cavity 20 to the third internal cavity 61 so that the temperature of the sensor element 101 can be adjusted to such a temperature that activates the solid electrolyte. The temperature may be adjusted so that the main pump cell 21, the first measurement pump cell 50, and the second measurement pump cell 41 are operable. It is not necessary that the whole area is adjusted to the same temperature, but the sensor element 101 may have temperature distribution. The sensor element 101 (base part 102) may have temperature distribution in the longitudinal direction, and may have temperature distribution in a thickness direction and/or a width direction. For example, the sensor element 101 may be heated so that temperature of the solid electrolyte around the measurement-object gas flow cavity 15 and the respective electrodes becomes at about 750° C. to about 900° C.

In the sensor element 101 of the present embodiment, the heater 72 is embedded in the base part 102, but this form is not limitative. The heater 72 may be disposed to heat the base part 102. That is, the heater 72 may heat the sensor element 101 to develop oxygen ion conductivity with which the main pump cell 21, the first measurement pump cell 50, and the second measurement pump cell 41 are operable. For example, the heater 72 may be embedded in the base part 102 as in the present embodiment. Alternatively, for example, the heater part 70 may be formed as a heater substrate that is separate from the base part 102, and may be disposed at a position adjacent to the base part 102.

The heater insulating layer 74 is formed of an insulator such as alumina on the upper and lower surfaces of the heater 72 and the heater lead 76. The heater insulating layer 74 is formed to ensure electrical insulation between the second substrate layer 2, and the heater 72 and the heater lead 76, and electrical insulation between the third substrate layer 3, and the heater 72 and the heater lead 76.

The pressure relief vent 75 extends through the third substrate layer 3 so that the heater insulating layer 74 and the reference gas introduction space 43 communicate with each other. The pressure relief vent 75 can mitigate an increase in internal pressure due to temperature rise in the heater insulating layer 74. The pressure relief vent 75 may be absent.

Further, an area of a predetermined length in the longitudinal direction in a surface of the sensor element 101 from the front end may be covered by a porous protective layer not shown. The porous protective layer is formed to protect a region where the internal cavities and the electrodes are present in the sensor element 101 from thermal shock caused by water splashing or the like. The porous protective layer may be made of ceramics such as alumina, and may have a thickness of approximately 10 μm to 2000 μm. Preferably, the porous protective layer may be formed so as to resist forces up to approximately 50 N.

The above-described sensor element 101 is incorporated into the gas sensor 100 in such a form that the front end part of the sensor element 101 comes into contact with the measurement-object gas, and the rear end part of the sensor element 101 comes into contact with the reference gas.

### (Control Unit)

The gas sensor 100 of this embodiment includes the sensor element 101 described above and the control unit 90 for controlling the sensor element 101. In the gas sensor 100, each of the electrodes 22, 23, 51, 44, and 42 of the sensor element 101 is electrically connected to the control unit 90 through a lead wire not shown. FIG. 2 is a block diagram showing electric connections between the control unit 90, and the respective pump cells 21, 50 and 41 and the respective sensor cells 80, 81, 82, and 83 of the sensor element 101. The control unit 90 includes the above-described variable power supplies 24, 52 and 46, and a control part 91. The control part 91 includes a pump control part 92 and a concentration calculating part 93.

The control part 91 is realized by a general-purpose or dedicated computer, and functions as the pump control part 92 and the concentration calculating part 93 are realized by a CPU, a memory or the like installed in the computer. It is to be noted that when water vapor H₂O and carbon dioxide CO₂ contained in exhaust gas from the engine of a car is target gases to be measured by the gas sensor 100 and the sensor element 101 is attached to an exhaust gas path, some or all of the functions of the control unit 90 (especially, the control part 91) may be realized by an electronic control unit (ECU) installed in the car.

The control part 91 is configured to acquire an electromotive force (voltage V0, V1, V2, Vref) in each of the sensor cells 80, 81, 82, and 83, and a pump current (Ip0, Ip1, Ip2) in each of the pump cells 21, 50 and 41 of the sensor element 101. Further, the control part 91 is configured to output control signals to the variable power supplies 24, 52 and 46.

The pump control part 92 is configured to control the operation of the oxygen pump cell (in this embodiment, the main pump cell 21), the first measurement pump cell 50, and the second measurement pump cell 41 so as to measure a water vapor concentration and a carbon dioxide concentration in a measurement-object gas.

The pump control part 92 applies a predetermined oxygen pump voltage (pump voltage Vp0) between the intracavity oxygen pump electrode (the inner main pump electrode 22) and the extracavity oxygen pump electrode (the outer pump electrode 23) of the oxygen pump cell (the main pump cell 21) to pump out oxygen from the first internal cavity 20 and adjust an oxygen partial pressure in the first internal cavity 20 such that substantially all of water vapor and carbon dioxide in the measurement-object gas are decomposed in the first internal cavity 20;
the pump control part 92 applies a predetermined first measurement pump voltage (pump voltage Vp1) between the intracavity first measurement electrode (the first measurement electrode 51) and the extracavity first measurement electrode (the outer pump electrode 23) of the first measurement pump cell 50 to pump oxygen into the second internal cavity 40 and adjust an oxygen partial pressure in the second internal cavity 40 such that hydrogen generated by decomposition of the water vapor in the first internal cavity 20 and introduced into the second internal cavity 40 is oxidized in the second internal cavity 40; and
the pump control part 92 applies a predetermined second measurement pump voltage (pump voltage Vp2) between the intracavity second measurement electrode (the second measurement electrode 44) and the extracavity second measurement electrode (the outer pump electrode 23) of the second measurement pump cell 41 to pump oxygen into the third internal cavity 61, and adjust an oxygen partial pressure in the third internal cavity 61 such that hydrogen generated by the decomposition of the water vapor in the first internal cavity 20 and introduced into the third internal cavity 61, and, carbon monoxide generated by decomposition of the carbon dioxide in the first internal cavity 20 and introduced into the third internal cavity 61 are oxidized in the third internal cavity 61.

The pump control part 92 may adjust the oxygen partial pressure in each of the internal cavities 20, 40, and 61 by adjusting the pump voltage (Vp0, Vp1, Vp2) applied to each of the pump cells 21, 50, and 41. Further, the pump control part 92 may adjust the oxygen partial pressure in each of the internal cavities 20, 40, and 61 by performing, for example, the following feedback control based on the electromotive force (voltage V0, V1, V2) generated in each of the sensor cells 80, 81, and 82. Specifically, in the present embodiment, control is performed in the following manner.

The pump control part 92 performs feedback control of the pump voltage Vp0 of the variable power supply 24 in the main pump cell 21 so that the electromotive force (the voltage V0) generated between the intracavity oxygen pump electrode (the inner main pump electrode 22) and the reference electrode 42 is at a constant value (referred to as a set value V0_{SET}). The set value V0_{SET} may be set as a value such that the oxygen partial pressure of the atmosphere in the first internal cavity 20 is made to be an oxygen partial pressure at which all of, or substantially all of the water vapor and the carbon dioxide in the measurement-object gas is decomposed in the first internal cavity 20. The set value V0_{SET} may vary depending on the intended use of the gas sensor 100, the configuration of the sensor element 101 and the like, and the set value V0_{SET} may be, for example, about 1200 mV or more and about 1500 mV or less, or, about 1300 mV or more and about 1500 mV or less. The set value V0_{SET} may be set such that the value increases as a concentration of the target gas to be measured (e.g., carbon dioxide concentration) in the measurement-object gas becomes higher.

The pump control part 92 performs feedback control of the pump voltage Vp1 of the variable power supply 52 in the first measurement pump cell 50 so that the electromotive force (the voltage V1) generated between the intracavity first measurement electrode (the first measurement electrode 51) and the reference electrode 42 is at a constant value (referred to as a set value V1_{SET}). The set value V1_{SET} may be set as a value such that the oxygen partial pressure of the atmosphere in the second internal cavity 40 is made to be an oxygen partial pressure at which a part of the hydrogen generated by the decomposition of the water vapor, that is, hydrogen introduced into the second internal cavity 40 of the hydrogen generated by the decomposition of the water vapor, is selectively burned in the second internal cavity 40. That is, the set value V1_{SET} may be set as a value such that the oxygen partial pressure of the atmosphere in the second internal cavity 40 is made to be an oxygen partial pressure at which all of, or substantially all of the hydrogen introduced into the second internal cavity 40 of the hydrogen generated by the decomposition of the water vapor is burned in the vicinity of the surface of the intracavity first measurement electrode (the first measurement electrode 51) in the second internal cavity 40, and the carbon monoxide generated by the decomposition of the carbon dioxide is not burned, or not substantially burned in the vicinity of the surface of the intracavity first measurement electrode (the first measurement electrode 51) in the second internal cavity 40. The phrase “not substantially burned” includes that burning of the carbon monoxide at the intracavity first measurement electrode (the first measurement electrode 51) is so low as not to affect measurement of a water vapor concentration and a carbon dioxide concentration in the gas sensor 100. The set value V1_{SET} may vary depending on the intended use of the gas sensor 100, the configuration of the sensor element 101 and the like, and the set value V1_{SET} may be, for example, about 300 mV or more and about 700 mV or less. Alternatively, the set value V1_{SET} may be, for example, about 350 mV or more, or about 400 mV or more, and may be, for example, about 650 mV or less, or about 600 mV or less.

The pump control part 92 performs feedback control of the pump voltage Vp2 of the variable power supply 46 in the second measurement pump cell 41 so that the electromotive force (the voltage V2) generated between the intracavity second measurement electrode (the second measurement electrode 44) and the reference electrode 42 is at a constant value (referred to as a set value V2_{SET}). The set value V2_{SET} may be set as a value such that the oxygen partial pressure of the atmosphere in the third internal cavity 61 is made to be an oxygen partial pressure at which the residual of the hydrogen generated by the decomposition of the water vapor, that is, hydrogen introduced into the third internal cavity 61 of the hydrogen generated by the decomposition of the water vapor, and, the carbon monoxide generated by the decomposition of the carbon dioxide is burned in the third internal cavity 61. That is, the set value V2_{SET} may be set as a value such that the oxygen partial pressure in the third internal cavity 61 is made to be an oxygen partial pressure at which all of, or substantially all of the hydrogen introduced into the third internal cavity 61 of the hydrogen generated by the decomposition of the water vapor, and, the carbon monoxide generated by the decomposition of the carbon dioxide are burned in the third internal cavity 61. The set value V2_{SET} is generally a value smaller than the set value V1_{SET}. The set value V2_{SET} may vary depending on the intended use of the gas sensor 100, the configuration of the sensor element 101 and the like, and the set value V2_{SET} may be, for example, about 150 mV or more and about 400 mV or less. The set value V2_{SET} may be, for example, about 200 mV.

The concentration calculating part 93 is configured to calculate a water vapor concentration and a carbon dioxide concentration in a measurement-object gas.

The concentration calculating part 93 calculates a water vapor concentration in the measurement-object gas based on a current (pump current Ip1) flowing through the first measurement pump cell 50; and
the concentration calculating part 93 calculates a carbon dioxide concentration in the measurement-object gas based on the current (pump current Ip1) flowing through the first measurement pump cell 50, and a current (pump current Ip2) flowing through the second measurement pump cell 41.

In this embodiment, the concentration calculating part 93 is configured to calculate the water vapor concentration in the measurement-object gas based on the current (pump current Ip1) flowing through the first measurement pump cell 50; and
calculate the carbon dioxide concentration in the measurement-object gas based on the water vapor concentration calculated based on the current (pump current Ip1) flowing through the first measurement pump cell 50, and the current (pump current Ip2) flowing through the second measurement pump cell 41.

The concentration calculating part 93 acquires the pump current Ip1 in the first measurement pump cell 50, and calculates H₂O concentration in the measurement-object gas on the basis of a previously-stored conversion parameter (Ip1-H₂O concentration conversion parameter) between the pump current Ip1 and the water vapor concentration (H₂O concentration) in the measurement-object gas. The concentration calculating part 93 outputs the calculated H₂O concentration as a measurement value of the gas sensor 100. The Ip1-H₂O concentration conversion parameter is previously stored, as data showing a relationship between the pump current Ip1 and the H₂O concentration in the measurement-object gas, in the memory of the control part 91 which functions as the concentration calculating part 93. The Ip1-H₂O concentration conversion parameter may appropriately be determined by those skilled in the art by, for example, previously performing an experiment on the gas sensor 100. The Ip1-H₂O concentration conversion parameter may be, for example, the coefficient of an approximate expression (e.g., linear function) obtained by experiment or a map showing the relationship between the pump current Ip1 and the H₂O concentration in the measurement-object gas. The Ip1-H₂O concentration conversion parameter may be specific to each individual gas sensor 100 or may be common to a plurality of gas sensors.

The concentration calculating part 93 acquires the pump current Ip2 in the second measurement pump cell 41, and calculates CO₂ concentration in the measurement-object gas on the basis of the H₂O concentration calculated above, and a previously-stored conversion parameter (Ip2-H₂O concentration-CO₂ concentration conversion parameter) among the pump current Ip2, and the water vapor concentration (H₂O concentration) and the carbon dioxide concentration (CO₂ concentration) in the measurement-object gas. The concentration calculating part 93 outputs the calculated CO₂ concentration as a measurement value of the gas sensor 100. The Ip2-H₂O concentration-CO₂ concentration conversion parameter is previously stored, as data showing a relationship among the pump current Ip2, and the H₂O concentration and the CO₂ concentration in the measurement-object gas, in the memory of the control part 91 which functions as the concentration calculating part 93. The Ip2-H₂O concentration-CO₂ concentration conversion parameter may appropriately be determined by those skilled in the art by, for example, previously performing an experiment on the gas sensor 100. The Ip2-H₂O concentration-CO₂ concentration conversion parameter may be, for example, the coefficient of an approximate expression (e.g., linear function) obtained by experiment or a map showing the relationship among the pump current Ip2, and the H₂O concentration and the CO₂ concentration in a measurement-object gas. The Ip2-H₂O concentration-CO₂ concentration conversion parameter may be specific to each individual gas sensor 100 or may be common to a plurality of gas sensors.

The concentration calculating part 93 may be configured to further calculate an oxygen concentration in the measurement-object gas based on a current (pump current Ip0) flowing through the oxygen pump cell (namely, the main pump cell 21), the current (pump current Ip1) flowing through the first measurement pump cell 50, and the current (pump current Ip2) flowing through the second measurement pump cell 41.

For example, the concentration calculating part 93 may calculate the oxygen concentration in the measurement-object gas based on the current (pump current Ip0) flowing through the oxygen pump cell (the main pump cell 21), the water vapor concentration calculated based on the current (pump current Ip1) flowing through the first measurement pump cell 50, and the carbon dioxide concentration calculated based on the current (pump current Ip1) flowing through the first measurement pump cell 50 and the current (pump current Ip2) flowing through the second measurement pump cell 41.

Specifically, for example, the concentration calculating part 93 may acquire the pump current Ip0 in the main pump cell 21, and may calculate O₂ concentration in the measurement-object gas on the basis of the H₂O concentration and CO₂ concentration calculated above, and a previously-stored conversion parameter (current-O₂ concentration conversion parameter) among the pump current Ip0, and the water vapor concentration (H₂O concentration), the carbon dioxide concentration (CO₂ concentration) and the oxygen concentration (O₂ concentration) in the measurement-object gas. The concentration calculating part 93 may output the calculated O₂ concentration as a measurement value of the gas sensor 100. The current-O₂ concentration conversion parameter is previously stored, as data showing a relationship among the pump current Ip0, and the H₂O concentration, the CO₂ concentration and the O₂ concentration in the measurement-object gas, in the memory of the control part 91 which functions as the concentration calculating part 93. The current-O₂ concentration conversion parameter may appropriately be determined by those skilled in the art by, for example, previously performing an experiment on the gas sensor 100. The current-O₂ concentration conversion parameter may be, for example, the coefficient of an approximate expression (e.g., linear function) obtained by experiment or a map showing the relationship among the pump current Ip0, and the H₂O concentration, the CO₂ concentration and the O₂ concentration in a measurement-object gas. The current-O₂ concentration conversion parameter may be specific to each individual gas sensor 100 or may be common to a plurality of gas sensors.

### [Water Vapor Detecting Current and Carbon Dioxide Detecting Current]

The water vapor detecting current (namely, the pump current Ip1) and the carbon dioxide detecting current (namely, the pump current Ip2) will be described in detail, referring to a conventional gas sensor.

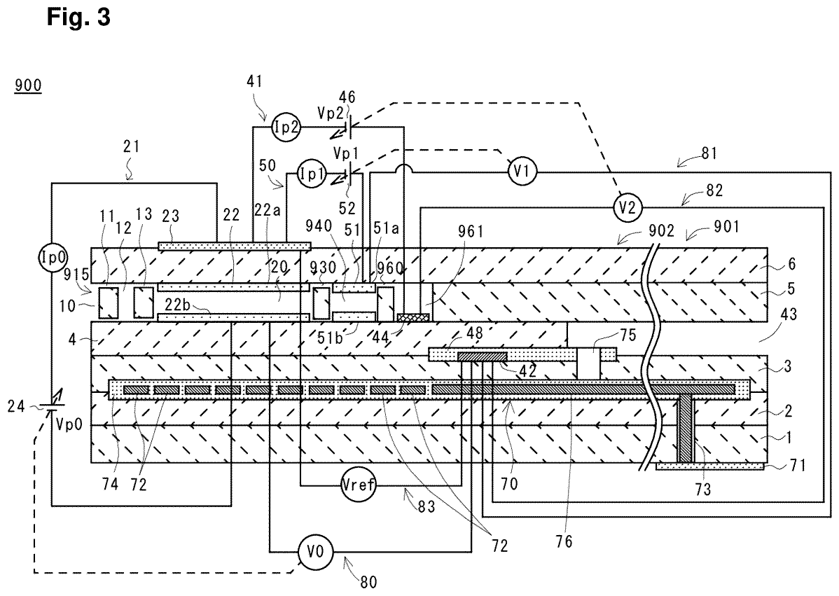

FIG. 3 is a vertical sectional schematic view in a longitudinal direction of a sensor element 901, showing one example of a schematic configuration of a gas sensor 900 of a comparative form. The gas sensor 900 is an example of a conventional gas sensor capable of measuring a water vapor concentration and a carbon dioxide concentration. In FIG. 3, the same member as in FIG. 1 is denoted by the same sign. In the sensor element 901, a measurement-object gas flow cavity 915 is formed in such a form that the first diffusion-rate limiting path 11, the buffer space 12, the auxiliary diffusion-rate limiting path 13, the first internal cavity 20, the second diffusion-rate limiting path 930, the second internal cavity 940, the third diffusion-rate limiting path 960, and the third internal cavity 961 communicate in this order in the longitudinal direction from the gas inlet 10. While the second internal cavity 40 and the third internal cavity 61 are formed independently in parallel in the above-described sensor element 101, the second internal cavity 940 and the third internal cavity 961 are formed to communicate in series in the sensor element 901. Therefore, the sensor element 901 is configured so that all of the measurement-object gas from the first internal cavity 20 reaches the third internal cavity 961 through the second internal cavity 940.

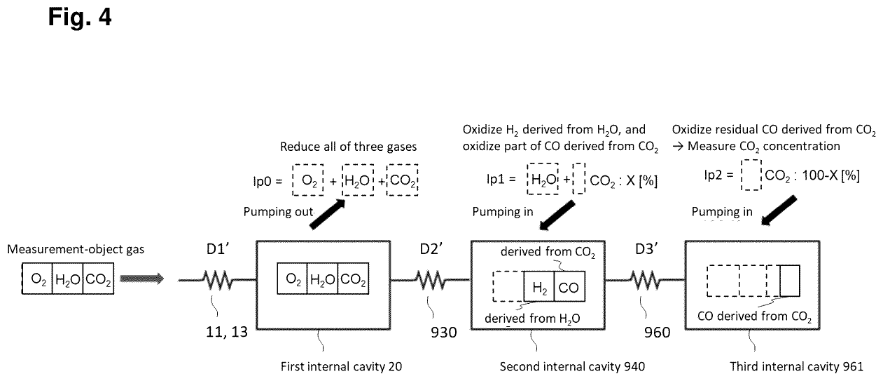

FIG. 4 is a conceptual diagram schematically showing a measurement principle of the gas sensor 900 and the behavior of the measurement-object gas in the respective internal cavities 20, 940, and 961. As gas species in the measurement-object gas, oxygen O₂, water vapor H₂O and carbon dioxide CO₂ that are related to measurement are illustrated. In FIG. 4, each diffusion-rate limiting path that functions as a diffusion resistance is indicated using a resistance symbol, and each of the internal cavities is indicated by a square. In each of the internal cavities, the gas introduced into said internal cavity is illustrated.

The behavior of the measurement-object gas in the gas sensor 900 will be explained by referring to FIG. 3 and FIG. 4. In the gas sensor 900, the measurement-object gas is introduced into the first internal cavity 20 via a diffusion resistance D1′ imparted by the first diffusion-rate limiting path 11 and the auxiliary diffusion-rate limiting path 13. In the first internal cavity 20, all of O₂, H₂O and CO₂ are reduced, and all of O₂ gas and oxygen generated by the reduction (or, decomposition) of H₂O and CO₂ are pumped out as the pump current Ip0. Thus, the pump current Ip0 is affected by all of O₂, H₂O and CO₂. Hydrogen H₂ and carbon monoxide CO generated by the reduction (or, decomposition) of H₂O and CO₂, respectively, are introduced into the second internal cavity 940 via a diffusion resistance D2′ imparted by the second diffusion-rate limiting path 930. In the second internal cavity 940, oxygen is pumped in as the pump current Ip1 to oxidize H₂. At that time, a part of CO (X % in FIG. 4) may also be oxidized. When the part of CO is oxidized, the pump current Ip1 is to depend not only on the water vapor concentration, but also on the carbon monoxide concentration. The residual CO (100−X % in FIG. 4) that has not been oxidized in the second internal cavity 940 is introduced into the third internal cavity 961 via a diffusion resistance D3′ imparted by the third diffusion-rate limiting path 960. In the third internal cavity 961, oxygen is pumped in as the pump current Ip2 to oxidize the residual CO (100−X % in FIG. 4). The pump current Ip2 depends on the carbon monoxide concentration. When the part of CO is oxidized in the second internal cavity 940, an amount of CO detected as the pump current Ip2 decreases, and this is considered to be disadvantageous in terms of measurement accuracy of the CO₂ concentration. Therefore, ideally, it is preferred that CO is not oxidized (or, an oxidation amount is at 0%) in the second internal cavity 940.

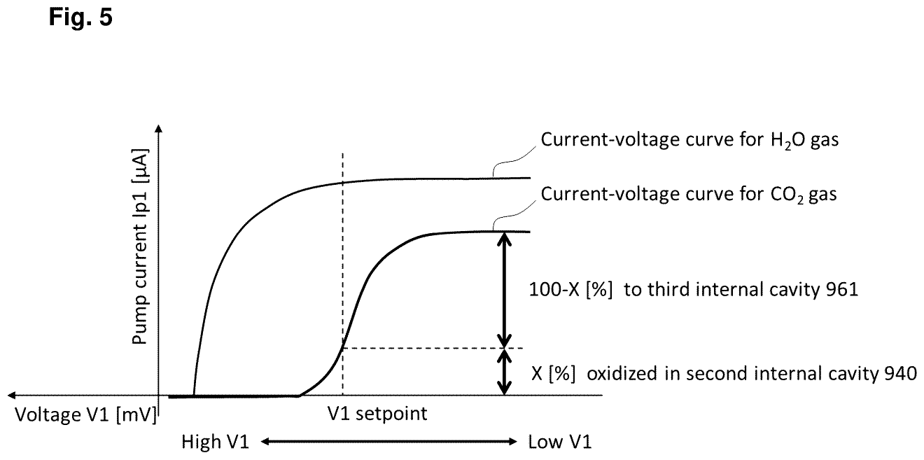

Factors causing a part of CO to be oxidized along with H₂ in the second internal cavity 940 are considered by referring to FIG. 5. FIG. 5 is a schematic diagram showing a relationship between the pump current Ip1 flowing through the first measurement pump cell 50, and the voltage V1 between the first measurement electrode 51 and the reference electrode 42. The horizontal axis of the FIG. 5 represents the voltage V1 [mV], with the voltage increasing from right to left. The vertical axis of FIG. 5 represents the pump current Ip1 [μA]. A current-voltage curve for H₂O gas indicates a current-voltage curve when a gas that contains water vapor and does not contain carbon dioxide is used as the measurement-object gas. The current-voltage curve for H₂O gas indicates a current flowing by hydrogen generated by reduction of the water vapor being oxidized on the first measurement electrode 51 of the first measurement pump cell 50. A current-voltage curve for CO₂ gas indicates a current-voltage curve when a gas that contains carbon dioxide and does not contain water vapor is used as the measurement-object gas. The current-voltage curve for CO₂ gas indicates a current flowing by carbon monoxide generated by reduction of the carbon dioxide being oxidized on the first measurement electrode 51 of the first measurement pump cell 50.

As shown in FIG. 5, in each of the current-voltage curve for H₂O gas and the current-voltage curve for CO₂, when the voltage V1 is decreased, the pump current Ip1 starts to flow at a certain voltage V1, and increases as the voltage V1 is decreased. When the voltage V1 becomes further low, the pump current Ip1 does not vary even when the voltage V1 is varied, and becomes to be saturated. This saturated current is referred to as a limiting current.

As shown in FIG. 5, in the current-voltage curve for H₂O gas, the pump current Ip1 starts to flow at a higher voltage V1 and becomes at the limiting current at a higher voltage V1, compared with the case of the current-voltage curve for CO₂. However, in a region in which the pump current Ip1 for H₂O gas is at the limiting current, the pump current Ip1 in the current-voltage curve for CO₂ has started to flow. That is, when a set value of the voltage V1 is determined so that the pump current Ip1 for H₂O gas is at the limiting current, a part (X %) of carbon monoxide generated by the reduction of the carbon dioxide is to be oxidized on the first measurement electrode 51 of the first measurement pump cell 50. It is to be noted that the current-voltage curve for H₂O gas and the current-voltage curve for CO₂ are the schematic diagrams for the above explanation, and does not indicate the magnitude of the pump currents Ip1, or values themselves of the pump currents Ip1 and the voltages V1.

In order to measure the water vapor concentration with higher accuracy based on the pump current Ip1, it is preferred to control the first measurement pump cell 50 so that the pump current Ip1 does not change in a current value with respect to the change in the voltage V1, that is, becomes a limiting current. Therefore, it is preferred that the voltage V1 is to be lower (or, the set value is to be smaller). On the other hand, in order to measure the carbon dioxide concentration with higher accuracy based on the pump current Ip2, it is preferred to control the first measurement pump cell 50 so that CO is not oxidized in the second internal cavity 940. Therefore, it is preferred that the voltage V1 is to be higher (or, the set value is to be larger). Accordingly, in the gas sensor 900, it is considered that there is a so-called trade-off relationship between the high precision measurement of the water vapor concentration and the high precision measurement of the carbon dioxide concentration.

The present inventor has found that by forming the second internal cavity 40 and the third internal cavity 61 independently in parallel as the above-described gas sensor 100, it is possible to get out of the trade-off relationship as described above, and the high precision measurement of the water vapor concentration and the high precision measurement of the carbon dioxide concentration can be compatible.

### [Measurement of Water Vapor Concentration and Carbon Dioxide Concentration]

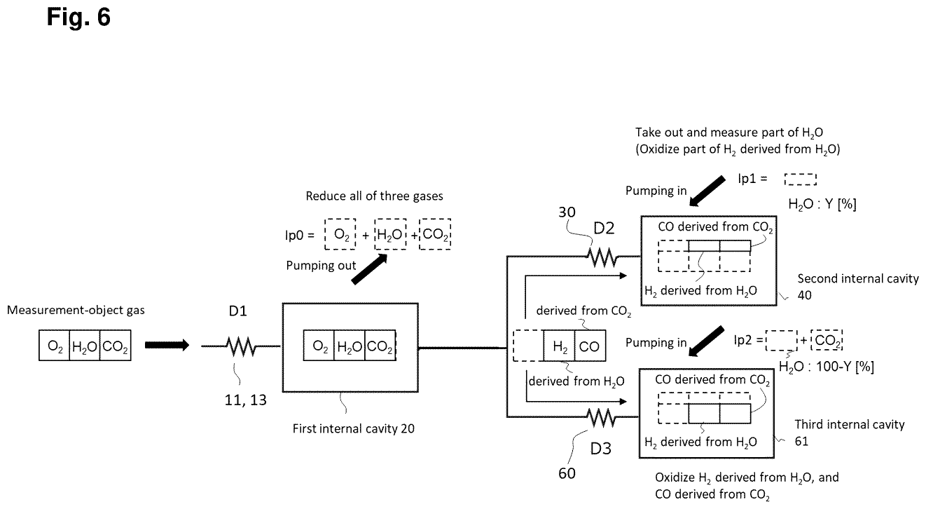

Next, a method for measuring each concentration of water vapor H₂O and carbon dioxide CO₂ in the measurement-object gas by using the gas sensor 100 having such a configuration as described above will be described. FIG. 6 is a conceptual diagram schematically showing a measurement principle of the gas sensor 100 and the behavior of the measurement-object gas in the respective internal cavities 20, 40, and 61. Also in FIG. 6, as in the case of FIG. 4, as gas species in the measurement-object gas, oxygen O₂, water vapor H₂O and carbon dioxide CO₂ that are related to measurement are illustrated. In FIG. 6, as in the case of FIG. 4, each diffusion-rate limiting path that functions as a diffusion resistance is indicated using a resistance symbol, and each of the internal cavities is indicated by a square. In each of the internal cavities, the gas introduced into said internal cavity is illustrated.

A control method of a gas sensor for measuring a concentration of each of water vapor H₂O and carbon dioxide CO₂ in a measurement-object gas includes:
a water vapor concentration measuring step of calculating a water vapor concentration in the measurement-object gas based on a current (pump current Ip1) flowing through the first measurement pump cell 50; and
a carbon dioxide concentration measuring step of calculating a carbon dioxide concentration in the measurement-object gas based on the current (pump current Ip1) flowing through the first measurement pump cell 50 and a current (pump current Ip2) flowing through the second measurement pump cell 41.

A method for measuring each concentration of water vapor H₂O and carbon dioxide CO₂ in the measurement-object gas by using the gas sensor 100 having such a configuration as described above, and the behavior of the measurement-object gas at that time will be explained by referring to FIG. 1 and FIG. 6.

The measurement-object gas is introduced from the gas inlet 10, passes through the first diffusion-rate limiting path 11, the buffer space 12, and the auxiliary diffusion-rate limiting path 13 in this order so that a predetermined diffusion resistance is imparted to the measurement-object gas, and reaches the first internal cavity 20. A diffusion resistance from the gas inlet 10 to being introduced into the first internal cavity 20 (mainly, a diffusion resistance of the first diffusion-rate limiting path 11 and a diffusion resistance of the auxiliary diffusion-rate limiting path 13) is represented as D1 in FIG. 6.

In the first internal cavity 20, by operating the main pump cell 21 as described above, the pump control part 92 pumps out oxygen from the first internal cavity 20 so that the oxygen partial pressure of the measurement-object gas introduced into the first internal cavity 20 is made to be a low oxygen partial pressure (for example, about 10⁻¹⁰ to 10⁻³⁰ atm) at which all of, or substantially all of water vapor and carbon dioxide contained in the measurement-object gas are decomposed. “All of, or substantially all of water vapor and carbon dioxide contained in the measurement-object gas are decomposed” means that the water vapor and the carbon dioxide introduced into the first internal cavity 20 are not substantially introduced into the second internal cavity 40.

When oxygen is pumped out from the first internal cavity 20 in such a manner, the decomposition reaction of water vapor (2H₂O→2H₂+O₂) and the decomposition reaction of carbon dioxide (2CO₂→2CO+O₂) are promoted in the first internal cavity 20 (especially, in the vicinity of the surface of the inner main pump electrode 22). Hydrogen and oxygen are generated by the decomposition of the water vapor, and carbon monoxide and oxygen are generated by the decomposition of the carbon dioxide. The generated oxygen is pumped out together with oxygen gas originally present in the measurement-object gas by the main pump cell 21. On the other hand, the hydrogen and the carbon monoxide are introduced into the second internal cavity 40.

The set value V0_{SET} of the voltage V0 may be preferably set such that an oxygen partial pressure (a target oxygen partial pressure) after adjusted in the first internal cavity 20 becomes smaller as the oxygen partial pressure in the measurement-object gas that has reached the first internal cavity 20 becomes larger. This may allow oxygen to be pumped out more reliably.

As shown in FIG. 6, the measurement-object gas, which contains the hydrogen generated by the decomposition of the water vapor and the carbon monoxide generated by the decomposition of the carbon dioxide, is distributed at a predetermined ratio and introduced into the second internal cavity 40 from the first internal cavity 20 via the second diffusion-rate limiting path 30, and into the third internal cavity 61 from the first internal cavity 20 via the third diffusion-rate limiting path 60, respectively.

That is, the measurement-object gas, which contains the hydrogen generated by the decomposition of the water vapor and the carbon monoxide generated by the decomposition of the carbon dioxide, passes through the second diffusion-rate limiting path 30 so that a predetermined diffusion resistance is imparted to the measurement-object gas, and reaches the second internal cavity 40. A diffusion resistance of the second diffusion-rate limiting path 30 is represented as D2 in FIG. 6. Further, the measurement-object gas, which contains the hydrogen generated by the decomposition of the water vapor and the carbon monoxide generated by the decomposition of the carbon dioxide, passes through the third diffusion-rate limiting path 60 so that a predetermined diffusion resistance is imparted to the measurement-object gas, and reaches the third internal cavity 61. A diffusion resistance of the third diffusion-rate limiting path 60 is represented as D3 in FIG. 6.

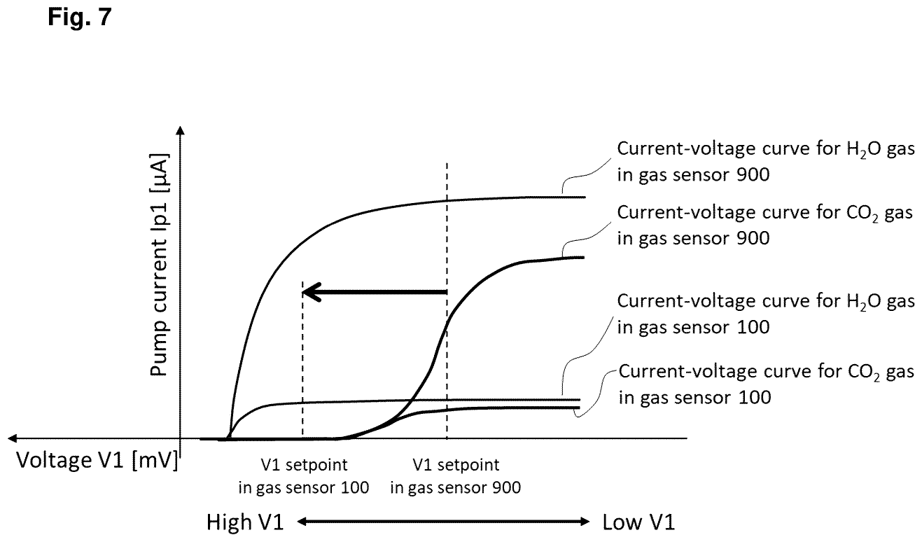

Thus, a predetermined part of the measurement-object gas, which contains the hydrogen generated by the decomposition of the water vapor and the carbon monoxide generated by the decomposition of the carbon dioxide, is to be introduced into the second internal cavity 40. Therefore, water vapor concentration may be measured using a predetermined part of the hydrogen generated by the decomposition of the water vapor. FIG. 7 is a schematic diagram showing a relationship between the pump current Ip1 flowing through the first measurement pump cell 50, and the voltage V1 between the first measurement electrode 51 and the reference electrode 42 as in the case of FIG. 5, and shows comparison between the gas sensor 100 of the embodiment according to the present invention and the gas sensor 900 of the example of the conventional gas sensor. As shown in FIG. 7, since an amount of hydrogen to be oxidized in the second internal cavity 40 can be decreased in the gas sensor 100, the pump current Ip1 due to the oxidation of the hydrogen can be made to reach a limiting current at a higher voltage V1. The pump current Ip1 due to the oxidation of the hydrogen is to be at the limiting current even when a set value of the voltage V1 is set to be higher, it is considered to be possible that substantially all of the hydrogen is oxidized in second internal cavity 40 without oxidizing carbon monoxide.

The predetermined ratio at which the measurement-object gas is distributed to the second internal cavity 40 and the third internal cavity 61 may vary depending on the intended use of the gas sensor 100, the configuration of the sensor element 101 and the like, and the predetermined ratio may be, for example, about 90% or less as a ratio of distributing to the second internal cavity 40. When the predetermined ratio is within such a range, it is possible to increase the voltage V1 at which the current due to the oxidation of hydrogen can be at a limiting current. Further, the ratio of distributing to the second internal cavity 40 may be, for example, about 80% or less, about 60% or less, about 50% or less, or about 25% or less. The predetermined ratio may be, for example, about 5% or more as the ratio of distributing to the second internal cavity 40. When the predetermined ratio is within such a range, it is possible to prevent the pump current Ip1 from being too small, and measurement with high accuracy can be achieved. Alternatively, the ratio of distributing to the second internal cavity 40 may be, for example, about 10% or more, or about 15% or more.

The predetermined ratio may be, for example, about 95% or less as a ratio of distributing to the third internal cavity 61. Further, the ratio of distributing to the third internal cavity 61 may be, for example, about 90% or less, or about 85% or less. The predetermined ratio may be, for example, about 10% or more as the ratio of distributing to the third internal cavity 61. Alternatively, the ratio of distributing to the third internal cavity 61 may be, for example, about 20% or more, about 40% or more, about 50% or more, or about 75% or more.

A ratio (distribution ratio) of an amount of the measurement-object gas distributed to the second internal cavity 40, to an amount of the measurement-object gas distributed to the third internal cavity 61 may be, for example, smaller than about 9. Alternatively, the distribution ratio may be smaller than about 7.25. The distribution ratio may be about 1 or less, or about 0.5 or less. A lower limit may be a value such that the pump current Ip1 does not become too small, and the distribution ratio may be, for example, about 0.05 or more, or about 0.08 or more.

As an index of the ratio (distribution ratio) of the amount of the measurement-object gas distributed to the second internal cavity 40 to the amount of the measurement-object gas distributed to the third internal cavity 61, a current ratio (Ip1/Ip2) of the current (namely, the pump current Ip1) flowing through the first measurement pump cell 50 to the current (namely, the pump current Ip2) flowing through the second measurement pump cell 41 when the measurement-object gas with the water vapor concentration of 10% and nitrogen as a remainder is introduced into the measurement-object gas flow cavity 15 may be used. For example, the gas sensor 100 is attached to a piping for measurement and driven, a model gas with the water vapor concentration of 10% and nitrogen as the remainder is made to flow through the piping for measurement, and the pump current Ip1 and the pump current Ip2 are measured. A ratio of the pump current Ip1 to the pump current Ip2 is calculated to obtain the current ratio (Ip1/Ip2).

The current ratio (Ip1/Ip2) may be, for example, smaller than about 7.25. When the predetermined ratio is within such a range, it is possible to increase the voltage V1 at which the current due to the oxidation of hydrogen can be at a limiting current. Further, the current ratio (Ip1/Ip2) may be, for example, about 1 or less, or about 0.5 or less. It is considered that the smaller the current ratio (Ip1/Ip2), the higher the voltage V1 at which the current due to the oxidation of the hydrogen can be at the limiting current tends to be. A lower limit may be a value such that the pump current Ip1 does not become too small, and the current ratio (Ip1/Ip2) may be, for example, about 0.05 or more, or about 0.08 or more. It is to be noted that water vapor concentration in the model gas for use of measuring the current ratio (Ip1/Ip2) does not need to be 10%, and may appropriately be determined within a range of about 3 to 30%. This is because roughly the same current ratio (Ip1/Ip2) is considered to be obtained regardless of the water vapor concentration.

The current ratio (Ip1/Ip2) can be at a desired value by appropriately setting the diffusion resistance D2 of the second diffusion-rate limiting path 30 and the diffusion resistance D3 of the third diffusion-rate limiting path 60. Generally, a diffusion resistance can be increased by making a width or a thickness of a diffusion-rate limiting path smaller, and/or making a length in the longitudinal direction longer. The dimensions of the second diffusion-rate limiting path 30 and the third diffusion-rate limiting path 60 may appropriately be determined so that the current ratio (Ip1/Ip2) becomes at the desired value.

For example, the diffusion resistance D2 of the second diffusion-rate limiting path 30 may be larger than the diffusion resistance D3 of the third diffusion-rate limiting path 60. This is considered to mean that the current ratio (Ip1/Ip2) is roughly less than one.

In the second internal cavity 40, by operating the first measurement pump cell 50 as described above, the pump control part 92 pumps oxygen into the second internal cavity 40. Consequently, hydrogen in the measurement-object gas containing hydrogen and carbon monoxide, which has reached the vicinity of the surface of the first measurement electrode 51 in the second internal cavity 40, selectively reacts with oxygen existing at this position for burning (2H₂+O₂→2H₂O). Oxygen may be pumped in by the first measurement pump cell 50 so that substantially all of the hydrogen having reached the vicinity of the surface of the first measurement electrode 51 is burned. As described above, the set value V1_{SET} may be, for example, about 300 mV or more and about 700 mV or less. When the set value V1_{SET} is within such a range, it is considered that substantially all of the hydrogen reached the vicinity of the surface of the first measurement electrode 51 is burned and the carbon monoxide reached the vicinity of the surface of the first measurement electrode 51 is not substantially burned. Since the carbon monoxide reached the vicinity of the surface of the first measurement electrode 51 is not oxidized at the first measurement electrode 51, it is considered that the carbon monoxide stays in the second internal cavity 40 unless the carbon dioxide concentration in the measurement-object gas changes and the carbon monoxide concentration in the second diffusion-rate limiting path 30 is varied.

The pump current Ip1 flowing through the first measurement pump cell 50 at this time is substantially proportional to an amount (or, a concentration) of the hydrogen burning in the vicinity of the surface of the first measurement electrode 51. An amount of hydrogen having reached the vicinity of the surface of the first measurement electrode 51 is correlated with a concentration of water vapor contained in the measurement-object gas introduced through the gas inlet 10. Therefore, by detecting the pump current Ip1, the water vapor concentration in the measurement-object gas can be calculated based on the detected value.

In the third internal cavity 61, by operating the second measurement pump cell 41 as described above, the pump control part 92 pumps oxygen into the third internal cavity 61. Consequently, both of hydrogen and carbon monoxide in the measurement-object gas containing the hydrogen and the carbon monoxide, which has reached the vicinity of the surface of the second measurement electrode 44 in the third internal cavity 61, react with oxygen existing at this position for burning (2H₂+O₂→2H₂O; 2CO+O₂→2CO₂). Oxygen may be pumped in by the second measurement pump cell 41 so that substantially all of the both hydrogen and carbon monoxide having reached the vicinity of the surface of the second measurement electrode 44 is burned.

The pump current Ip2 flowing through the second measurement pump cell 41 at this time relates to an amount (or, a concentration) of the hydrogen and an amount (or, a concentration) of the carbon monoxide both burning in the vicinity of the surface of the second measurement electrode 44. An amount of hydrogen having reached the vicinity of the surface of the second measurement electrode 44 is correlated with a concentration of water vapor contained in the measurement-object gas introduced through the gas inlet 10. An amount of carbon monoxide having reached the vicinity of the surface of the second measurement electrode 44 is correlated with a concentration of carbon dioxide contained in the measurement-object gas introduced through the gas inlet 10. Therefore, by using the pump current Ip2, and the pump current Ip1 or the water vapor concentration calculated from the pump current Ip1, the carbon dioxide concentration in the measurement-object gas can be calculated based on these values.

According to the measurement principle in FIG. 6, the gas sensor 100 ideally exhibits the following behavior depending on the gas composition of the measurement-object gas.

For example, when carbon dioxide concentration is varied under the absence of oxygen and water vapor,
the pump current Ip0 has a linear relationship with respect to the carbon dioxide concentration,
the pump current Ip1 is substantially at zero regardless of the carbon dioxide concentration, and
the pump current Ip2 has a linear relationship with respect to the carbon dioxide concentration.

For example, when water vapor concentration is varied under the absence of oxygen and carbon dioxide,
the pump current Ip0 has a linear relationship with respect to the water vapor concentration,
the pump current Ip1 has a linear relationship with respect to the water vapor concentration, and
the pump current Ip2 has a linear relationship with respect to the water vapor concentration.

For example, when water vapor concentration is varied in each of multiple carbon dioxide concentrations under the absence of oxygen,
the pump current Ip0 has a linear relationship with respect to the water vapor concentration in each of the carbon dioxide concentrations, and shifts upwards while maintaining the same slope of the linear relationship as the carbon dioxide concentration increases;
the pump current Ip1 has the same linear relationship with respect to the water vapor concentration regardless of the carbon dioxide concentration; and
the pump current Ip2 has a linear relationship with respect to the water vapor concentration in each of the carbon dioxide concentrations, and shifts upwards while maintaining the same slope of the linear relationship as the carbon dioxide concentration increases.

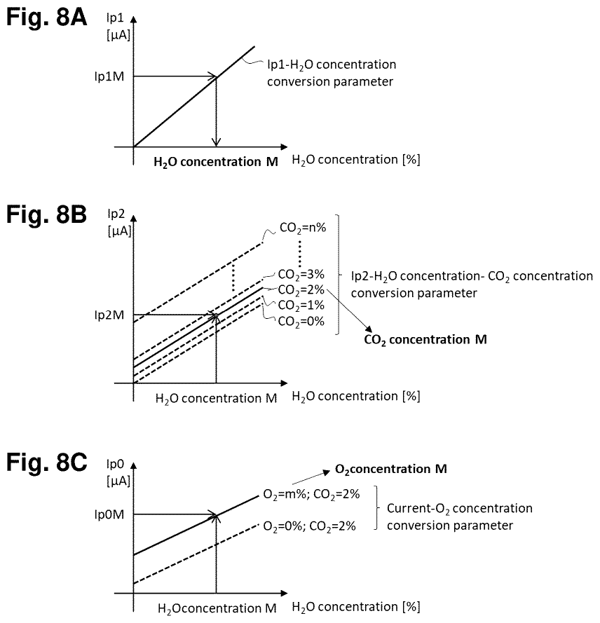

Next, a method for calculating a water vapor concentration and a carbon dioxide concentration by the concentration calculating part 93 will be described in detail. Here, a method for calculating an oxygen concentration will also be described. FIGS. 8A to 8C show schematic diagrams for explanation of the method for calculating the water vapor concentration, the carbon dioxide concentration, and the oxygen concentration by using the pump currents Ip1, Ip2 and Ip0. FIG. 8A is a schematic diagram for calculating the water vapor concentration (H₂O concentration). The horizontal axis of the diagram represents the H₂O concentration [%] and the vertical axis of the diagram represents the pump current Ip1 [μA]. FIG. 8B is a schematic diagram for calculating the carbon dioxide concentration (CO₂ concentration). The horizontal axis of the diagram represents the H₂O concentration [%] and the vertical axis of the diagram represents the pump current Ip2 [μA]. FIG. 8C is a schematic diagram for calculating the oxygen concentration (O₂ concentration). The horizontal axis of the diagram represents the H₂O concentration [%] and the vertical axis of the diagram represents the pump current Ip0 [μA].

As described above, the concentration calculating part 93 acquires the pump current Ip1 (in FIG. 8A, Ip1M; “M” means “Measured”. Hereinafter the same shall apply), and determines H₂O concentration M that corresponds to the Ip1M using the Ip1-H₂O concentration conversion parameter (FIG. 8A). As the Ip1-H₂O concentration conversion parameter, for example, a correlation expression between the H₂O concentration and the pump current Ip1, or the like may be previously stored. In the example of FIG. 8A, on the Ip1-H₂O concentration conversion parameter, a H₂O concentration when the pump current Ip1 is Ip1M is obtained as the H₂O concentration M.

Next, as described above, the concentration calculating part 93 acquires the pump current Ip2 (in FIG. 8B, Ip2M), and determines CO₂ concentration M that corresponds to the Ip2M, and the H₂O concentration M determined in FIG. 8A, using the Ip2-H₂O concentration-CO₂ concentration conversion parameter (FIG. 8B). As the Ip2-H₂O concentration-CO₂ concentration conversion parameter, for example, correlation expressions between the H₂O concentration and the pump current Ip2 in the respective multiple CO₂ concentrations, or the like may be previously stored. In the example of FIG. 8B, a correlation expression passing through the intersection point of Ip2M and the H₂O concentration M is determined among the correlation expressions between the H₂O concentration and the pump current Ip2 in the respective multiple CO₂ concentrations, and a CO₂ concentration in said correlation expression is obtained as the CO₂ concentration M. In the example of FIG. 8B, the CO₂ concentration M is 2%.

In FIG. 8B, a bundle of the correlation expressions between the H₂O concentration and the pump current Ip2 in the respective multiple CO₂ concentrations is used as the Ip2-H₂O concentration-CO₂ concentration conversion parameter. However, the Ip2-H₂O concentration-CO₂ concentration conversion parameter is not limited to this. The Ip2-H₂O concentration-CO₂ concentration conversion parameter may show the correspondence relationship among the pump current Ip2, the H₂O concentration and the CO₂ concentration. The Ip2-H₂O concentration-CO₂ concentration conversion parameter may be, for example, a bundle of the correlation expressions between the CO₂ concentration and the pump current Ip2 in the respective multiple H₂O concentrations, or a map showing the correspondence relationship among the pump current Ip2, the H₂O concentration and the CO₂ concentration.

As described above, the concentration calculating part 93 further acquires the pump current Ip0 (in FIG. 8C, Ip0M), and determines O₂ concentration M that corresponds to the Ip0M, the H₂O concentration M determined in FIG. 8A and the CO₂ concentration M determined in FIG. 8B, using the current-O₂ concentration conversion parameter (FIG. 8C). As the current-O₂ concentration conversion parameter, for example, correlation expressions between the H₂O concentration and the pump current Ip0 in the respective multiple CO₂ concentrations at each of multiple O₂ concentrations, or the like may be previously stored. In the example of FIG. 8C, a correlation expression passing through the intersection point of Ip0M and the H₂O concentration M is determined among the correlation expressions between the H₂O concentration and the pump current Ip0 when the CO₂ concentration is fixed at 2% at the multiple O₂ concentrations, and a O₂ concentration in said correlation expression is obtained as the O₂ concentration M.

In FIG. 8C, a bundle of the correlation expressions between the H₂O concentration and the pump current Ip0 in the respective multiple CO₂ concentrations at each of the multiple O₂ concentrations is used as the current-O₂ concentration conversion parameter. However, the current-O₂ concentration conversion parameter is not limited to this. The current-O₂ concentration conversion parameter may show the correspondence relationship among the pump current Ip0, the H₂O concentration, the CO₂ concentration and the O₂ concentration. The current-O₂ concentration conversion parameter may be, for example, a bundle of the correlation expressions between the CO₂ concentration and the pump current Ip0 in the respective multiple H₂O concentrations, or a map showing the correspondence relationship among the pump current Ip0, the H₂O concentration, the CO₂ concentration and the O₂ concentration.

### [Variation]

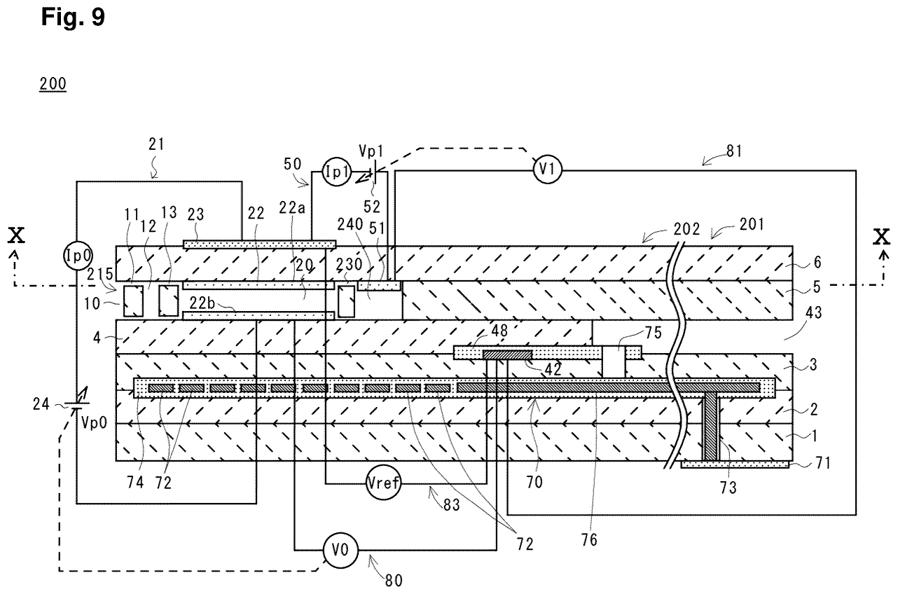

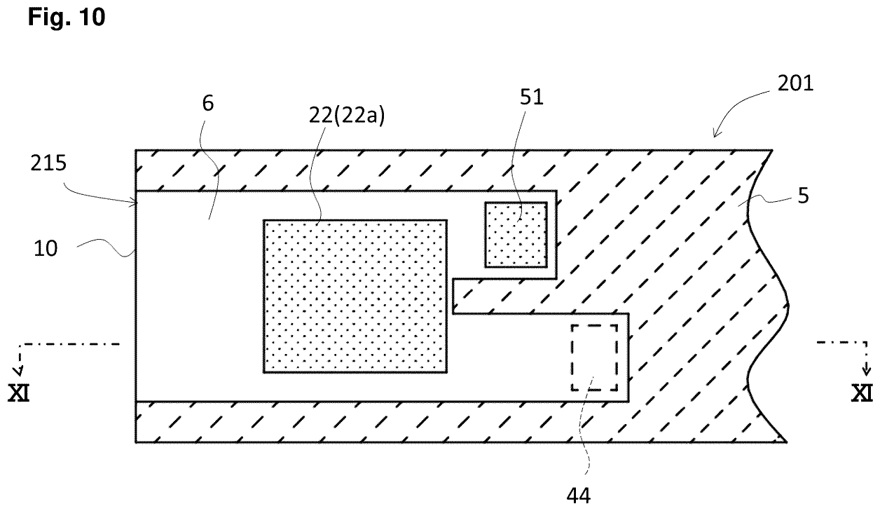

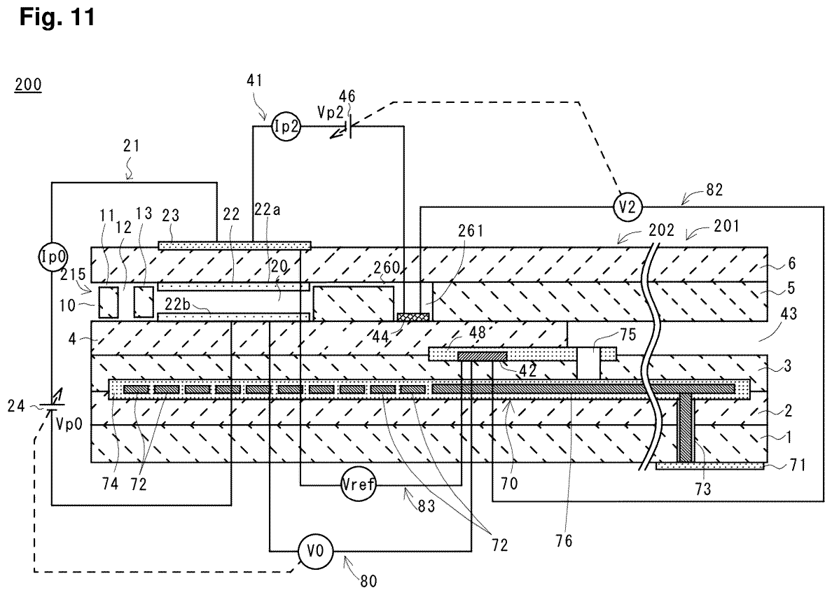

In the above-described embodiment, the second internal cavity 40 and the third internal cavity 61 are arranged vertically in parallel in the thickness direction. However, the present invention is not limited thereto. FIG. 9 is a vertical sectional schematic view in a longitudinal direction of a sensor element 201, showing one example of a general configuration of a gas sensor 200 including the sensor element 201. In FIG. 9, the same member as in FIG. 1 is denoted by the same sign. FIG. 10 is a partial sectional schematic view of the sensor element 201, showing a part of the section along line X-X in FIG. 9. FIG. 10 is a schematic view showing a general planar arrangement of the inner main pump electrode 22, the first measurement electrode 51, and the second measurement electrode 44. FIG. 11 is a vertical sectional schematic view in the longitudinal direction of the sensor element 201 along line XI-XI in FIG. 10, showing one example of the general configuration of the gas sensor 200 including the sensor element 201. FIG. 11 is the vertical sectional schematic view in the longitudinal direction at a position different from FIG. 9. That is, FIG. 9 is the vertical sectional schematic view in the longitudinal direction at a position where the first measurement electrode 51 is present in the sensor element 201, and FIG. 11 is the vertical sectional schematic view in the longitudinal direction at a position where the second measurement electrode 44 is present in the sensor element 201. As can be seen in FIG. 9 and FIG. 11, the second measurement electrode 44 is not present in the section of FIG. 10, but indicates by a dotted line in FIG. 10 to show the arrangement of the respective electrodes in the longitudinal direction of a base part 202. From each electrode toward the rear end of the element, an electrode lead which is not shown is disposed to allow connection with the outside. While FIG. 10 shows only the respective electrodes 22, 51, and 44 on the second solid electrolyte layer 6, the respective electrodes 22, 51, and 44 are disposed in the respective internal cavities 20, 240, and 261, and the diffusion-rate limiting path is interposed between internal cavities, as shown in FIG. 9 and FIG. 11.

In a measurement-object gas flow cavity 215 of the sensor element 201 (base part 202), a second diffusion-rate limiting path 230 and a second internal cavity 240, and a third diffusion-rate limiting path 260 and a third internal cavity 261 are arranged in parallel in a width direction. Other than the above, the gas sensor 200 is the same as the gas sensor 100, and thus all of the above descriptions regarding the gas sensor 100 are applicable to the gas sensor 200.

The gas sensor 100 and the gas sensor 200 have been described above as examples of the embodiment according to the present invention, but the present invention is not limited thereto. The present invention may include a gas sensor having any structure as long as the object of the present invention can be achieved, that is, a gas sensor that can accurately measure both of a water vapor concentration and a carbon dioxide concentration in a measurement-object gas is provided.

In the gas sensor 100 of the above embodiment, the intracavity second measurement electrode (the second measurement electrode 44) of the second measurement pump cell 41 is arranged at a rear end side rather than the intracavity first measurement electrode (the first measurement electrode 51) of the first measurement pump cell 50 in the longitudinal direction of the sensor element 101 (base part 102). However, the arrangement of the electrodes is not limited thereto. In the longitudinal direction of the sensor element 101 (base part 102), the second measurement electrode 44 may be present at the same position as the first measurement electrode 51, or may be arranged at a front end side rather than the first measurement electrode 51. The same is true for the gas sensor 200.

In the gas sensor 100 of the above embodiment, the inner main pump electrode 22 is composed of the ceiling electrode portion 22a formed on the ceiling surface of the first internal cavity 20, the bottom electrode portion 22b formed on the bottom surface of the first internal cavity 20, and the lateral electrode portions formed on the lateral wall surfaces of the first internal cavity 20 so as to connect the ceiling electrode portion 22a and the bottom electrode portion 22b. However, the inner main pump electrode 22 is not limited thereto. For example, the inner main pump electrode 22 may be formed only on the ceiling surface of the first internal cavity 20. Alternatively, the inner main pump electrode 22 may be formed only on the bottom surface of the first internal cavity 20. For example, when the inner main pump electrode 22 has the ceiling electrode portion 22a and the bottom electrode portion 22b, the ceiling electrode portion 22a and the bottom electrode portion 22b may be the same in size, or the size of the ceiling electrode portion 22a and the bottom electrode portion 22b may be different from each other. The same is true for the gas sensor 200.

In the gas sensor 100 of the above embodiment, the first measurement electrode 51 is formed on the ceiling surface of the second internal cavity 40. However, the first measurement electrode 51 is not limited thereto. The first measurement electrode 51 may be formed on the bottom surface of the second internal cavity 40. Alternatively, the first measurement electrode 51 may be in a tunnel-like structure similar to the inner main pump electrode 22. Further, the second measurement electrode 44 is formed on the bottom surface of the third internal cavity 61. However, the second measurement electrode 44 is not limited thereto. The second measurement electrode 44 may be formed on the ceiling surface of the third internal cavity 61. Alternatively, the second measurement electrode 44 may be in a tunnel-like structure similar to the inner main pump electrode 22. The same are true for the gas sensor 200.

In the gas sensor 100 and the gas sensor 200 of the above-described embodiments, the outer pump electrode 23 has three functions as an extracavity oxygen pump electrode in the oxygen pump cell (main pump cell 21), an extracavity first measurement electrode in the first measurement pump cell 50, and an extracavity second measurement electrode in the second measurement pump cell 41. However, the outer pump electrode 23 is not limited thereto. For example, the extracavity oxygen pump electrode, the extracavity first measurement electrode, and the extracavity second measurement electrode may be formed as different electrodes. For example, any one or more of the extracavity oxygen pump electrode, the extracavity first measurement electrode, and the extracavity second measurement electrode may be provided on the outer surface of the base part 102 separately from the outer pump electrode 23 so as to be in contact with a measurement-object gas. Alternatively, the reference electrode 42 may also serve as any one or more of the extracavity oxygen pump electrode, the extracavity first measurement electrode, and the extracavity second measurement electrode.

In the gas sensor 100 of the above-described embodiment, each of the first diffusion-rate limiting path 11, the auxiliary diffusion-rate limiting path 13, the second diffusion-rate limiting path 30 and the third diffusion-rate limiting path 60 is provided as slit-shaped path. However, the diffusion-rate limiting path is not limited thereto. The diffusion-rate limiting path may function as a diffusion resistance. Any one or more of the first diffusion-rate limiting path 11, and the auxiliary diffusion-rate limiting path 13, the second diffusion-rate limiting path 30 and the third diffusion-rate limiting path 60 may be composed of a porous body. Further, in the gas sensor 100 of the above-described embodiment, each of the second diffusion-rate limiting path 30 and the third diffusion-rate limiting path 60 is provided as a single slit between the ceiling surface and the wall surface. However, any one or more of the second diffusion-rate limiting path 30 and the third diffusion-rate limiting path 60 is provided as a single slit between the bottom surface and the wall surface. Alternatively, each or both of the second diffusion-rate limiting path 30 and the third diffusion-rate limiting path 60 may be provided as two slits similar to the first diffusion-rate limiting path 11 and the auxiliary diffusion-rate limiting path 13.

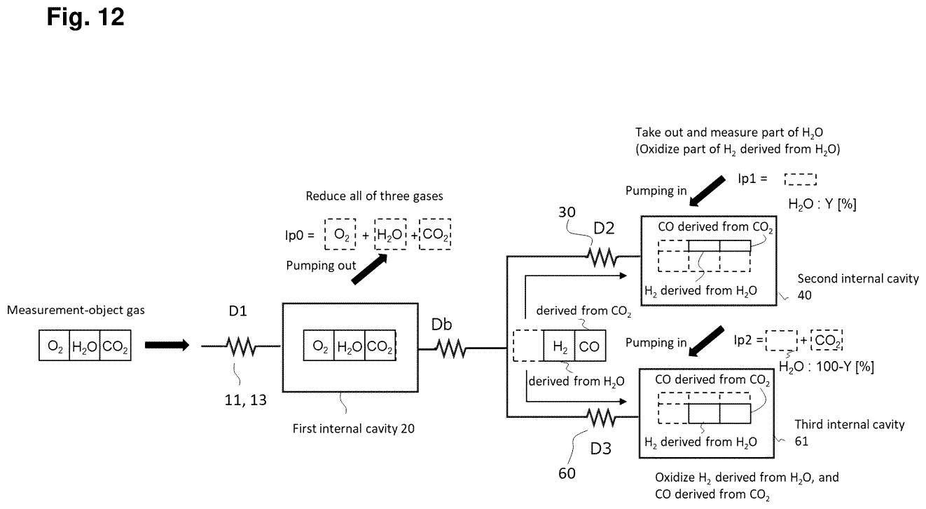

In the gas sensor 100 of the above-described embodiment, the second diffusion-rate limiting path 30 is formed between the first internal cavity 20 and the second internal cavity 40, and the third diffusion-rate limiting path 60 is formed between the first internal cavity 20 and the third internal cavity 61, as shown in FIG. 1 and FIG. 6. However, the configuration of the diffusion-rate limiting paths is not limited to this. For example, as shown in FIG. 12, a diffusion resistance Db may further be arranged between the first internal cavity 20, and the second diffusion-rate limiting path 30 and the third diffusion-rate limiting path 60. FIG. 12 is a conceptual diagram schematically showing a measurement principle of a gas sensor and the behavior of the measurement-object gas in the respective internal cavities 20, 40, and 61, as in the case of FIG. 6. In FIG. 12, the same member as in FIG. 6 is denoted by the same sign. The diffusion resistance Db may be, for example, a diffusion-rate limiting path that is formed rearward the first internal cavity 20 and communicates with both of the second diffusion-rate limiting path 30 and the third diffusion-rate limiting path 60.

FIGS. 1, and 9 to 11 have been shown above as examples of the embodiment according to the present invention, but each of FIGS. 1, and 9 to 11 is schematically illustrated for understanding the present invention. Dimensions, a scale, and arrangement of each of the components are not limited to FIGS. 1, and 9 to 11.

### [Method for Producing Gas Sensor]

Next, one example of a method for producing the gas sensor as described above is described. A plurality of unfired sheet moldings (so-called green sheets) containing an oxygen-ion-conductive solid electrolyte such as zirconia (ZrO₂) as a ceramic component are subjected to a predetermined processing and printing of circuit pattern, and then the plurality of sheets are laminated, and the laminate was cut, and then fired. Thus the sensor element 101 can be manufactured. Then, the manufactured sensor element may be incorporated into the gas sensor.

Hereinafter, description is made while taking the case of manufacturing the sensor element 101 composed of six layers shown in FIG. 1 as an example.

First, eight green sheets containing an oxygen-ion-conductive solid electrolyte such as zirconia (ZrO₂) as a ceramic component are prepared. For manufacturing of the green sheets, a known molding method can be used. The eight green sheets may all have the same thickness, or the thickness differs depending on the layer to be formed. In each of the eight green sheets, sheet holes or the like for use in positioning at the time of printing or stacking are formed in advance by a known method such as a punching process with a punching apparatus to prepare a blank sheet. In the blank sheets for use as the spacer layers 5a, 5b, and 5c, penetrating parts such as internal cavities are also formed in the same manner. Also in the remaining layers, necessary penetrating parts are formed in advance.

The blank sheets for use as six layers, namely, the first substrate layer 1, the second substrate layer 2, the third substrate layer 3, the first solid electrolyte layer 4, the spacer layer 5 (5a, 5b, and 5c), and the second solid electrolyte layer 6 are subjected to printing of various patterns required for respective layers and drying treatment. For printing of a pattern, a known screen printing technique can be used. Also as the drying treatment, a known drying means can be used.

After completing the printing and drying of diverse patterns for each of the eight blank sheets by repeating these steps, contact bonding treatment of stacking the eight printed blank sheets in a predetermined order while positioning with the sheet holes and the like, and contact bonding at a predetermined temperature and pressure condition to give a laminate is conducted.

The contact bonding treatment is conducted by heating and pressurizing with a known laminator such as a hydraulic press. While the temperature, the pressure and the time of heating and pressurizing depend on the laminator being used, they may be appropriately determined to achieve excellent lamination.

The obtained laminate includes a plurality of sensor elements 101. The laminate is cut into units of the sensor element 101. The cut laminate is fired at a predetermined firing temperature to obtain the sensor element 101. That is, the sensor element 101 is obtained by integral firing of the solid electrolyte layers and the electrodes. The firing temperature may be such a temperature that the solid electrolyte forming the base part 102 of the sensor element 101 is sintered to become a dense product, and an electrode or the like maintains desired porosity. The firing is conducted, for example, at a firing temperature of about 1200° C. or more and 1500° C. or less.

The obtained sensor element 101 is incorporated into the gas sensor 100 in such a form that the front end part of the sensor element 101 comes into contact with the measurement-object gas, and the rear end part of the sensor element 101 comes into contact with the reference gas.

### EXAMPLES

Hereinafter, Examples are described. The present invention is not limited to the following Examples.

Using the configuration shown in FIG. 12 as Example and the configuration of the sensor 900 shown in FIG. 4 as Comparative Example, the effect of the present invention was verified by simulation. COMSOL Multiphysics was used for the simulation.

Examples 1 to 5 were configured so that the second internal cavity 40 and the third internal cavity 61 were arranged vertically rearward of the first internal cavity 20. In each of Examples 1 to 5, the diffusion resistance D2 was 100 [cm⁻¹](Example 1), 500 [cm⁻¹](Example 2), 3000 [cm⁻¹](Example 3), 6000 [cm⁻¹](Example 4), or 9000 [cm⁻¹](Example 5). In all of Examples 1 to 5, the diffusion resistances other than the diffusion resistance D2 were the same. Specifically, the diffusion resistance D1 was 2716 [cm⁻¹], the diffusion resistance Db was 2500 [cm⁻¹], and the diffusion resistance D3 was 500 [cm⁻¹]. It is to be noted that in each diffusion-rate limiting path, a value of dividing a length in the longitudinal direction of the sensor element 101 of the diffusion-rate limiting path by a cross-sectional area of said diffusion-rate limiting path was defined as a diffusion resistance of said diffusion-rate limiting path. The diffusion resistance D1 was the sum of a diffusion resistance of the first diffusion-rate limiting path 11 and a diffusion resistance of the auxiliary diffusion-rate limiting path 13. In all of Examples 1 to 5, the arrangement and dimensions of the respective internal cavities 20, 40, and 61 and the respective electrodes 22, 23, 51, and 44 were the same, and all of other conditions for the simulation were also the same.

Comparative Example 1 was configured so that the first internal cavity 20, the second internal cavity 40 and the third internal cavity 61 were arranged in series as shown in FIG. 4. In Comparative Example 1, the diffusion resistance D1′ was 2716 [cm⁻¹], the diffusion resistance D2′ was 2500 [cm⁻¹], and the diffusion resistance D3′ was 500 [cm⁻¹]. The dimensions of the respective electrodes 22, 23, 51, and 44 were the same as Examples 1 to 5, and all of other conditions for the simulation were also the same as Examples 1 to 5.

First, only water vapor H₂O was used as a gas component, and a ratio (Ip1:Ip2) of the pump current Ip1 and the pump current Ip2 were obtained. H₂O was assumed to constitute 30% of the gas. The pump current Ip1 and the pump current Ip2 were derived when the set value V1_{SET} was 300 mV, and the ratio (Ip1:Ip2) was obtained. It is to be noted that the set value V0_{SET} was 1300 mV, and the set value V2_{SET} was 200 mV.

In Examples 1 to 5, the ratio (Ip1:Ip2) roughly indicates a ratio of the measurement-object gas being distributed in the second internal cavity 40 and the third internal cavity 61. In Comparative Example 1, the ratio (Ip1:Ip2) indicates a ratio of hydrogen oxidized in the second internal cavity 940 and hydrogen oxidized in the third internal cavity 961.

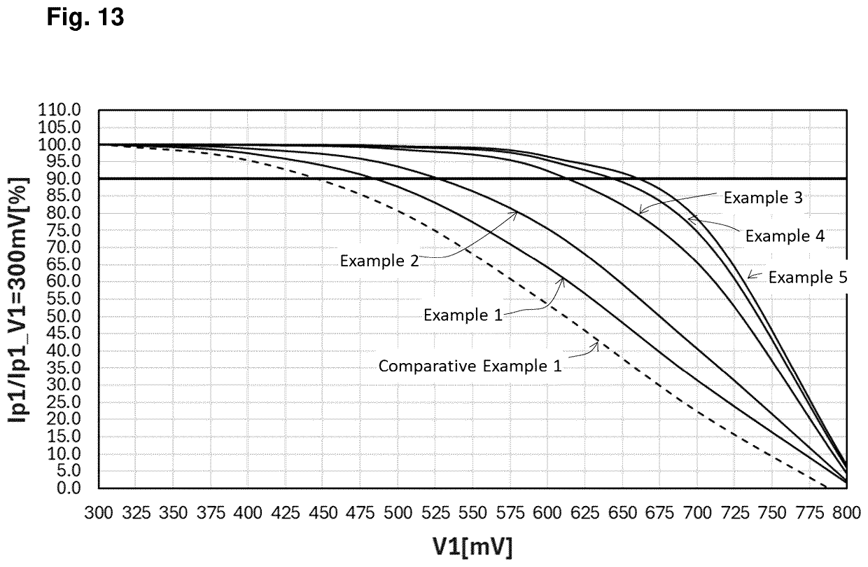

Further, only water vapor H₂O was used as a gas component, and a current voltage curve of the pump current Ip1 for H₂O was obtained in each of Examples 1 to 5 and Comparative Example 1. H₂O was assumed to constitute 30% of the gas. The set value V1_{SET} was varied from 300 mV to 800 mV, and the pump current Ip1 at each of the voltage V1 was obtained. FIG. 13 is a graph showing a current voltage curve of the pump current Ip1 for H₂O in each of Examples 1 to 5 and Comparative Example 1. The horizontal axis of the graph represents the voltage V1 [mV] and the vertical axis of the graph represents a relative value (Ip1/Ip1_V1=300 mV) [%] of the pump current Ip1 when the pump current Ip1 at the voltage V1 of 300 mV was set to 100%. It is considered that the pump current Ip1 when the voltage V1 is at 300 mV is to be a limiting current with respect to the water vapor. The relative value (Ip1/Ip1_V1=300 mV) of the pump current Ip1 being close to 100% even at the higher voltage V1 indicates that the pump current Ip1 becomes at the limiting current even at the higher voltage V1, which is more preferable from the perspective of measurement accuracy.

As an evaluation index 1, a voltage V1 [mV] when the relative value (Ip1/Ip1_V1=300 mV) of the pump current Ip1 was at 90% is shown in Table 1. It is considered that the effect of measuring a water vapor concentration at the limiting current and not oxidizing CO in the second internal cavity 40 becomes higher as the voltage V1 of the evaluation index 1 increases.

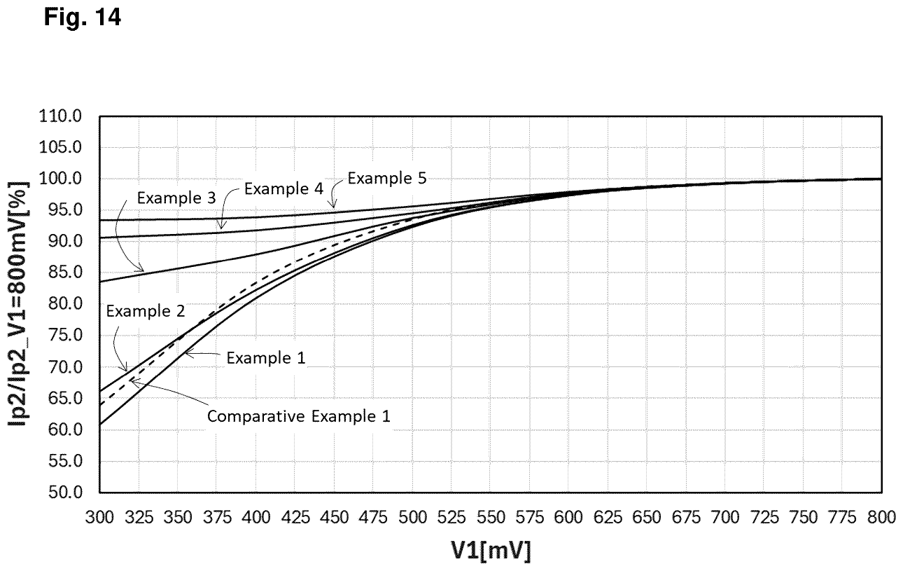

Next, only carbon dioxide CO₂ was used as a gas component, and a relationship of the voltage V1 between the first measurement electrode 51 and the reference electrode 44 with the pump current Ip2 in the second measurement pump cell 41 was obtained in each of Examples 1 to 5 and Comparative Example 1. CO₂ was assumed to constitute 30% of the gas. The set value V1_{SET} was varied from 300 mV to 800 mV and, the pump current Ip2 at each of the voltage V1 was obtained. FIG. 14 is a graph showing a relationship of the voltage V1 between the first measurement electrode 51 and the reference electrode 44 with the pump current Ip2 in the second measurement pump cell 41 in each of Examples 1 to 5 and Comparative Example 1. The horizontal axis of the graph represents the voltage V1 [mV] and the vertical axis of the graph represents a relative value (Ip2/Ip2_V1=800 mV) [%] of the pump current Ip2 when the pump current Ip2 at the voltage V1 of 800 mV was set to 100%. It is considered that when the voltage V1 is at 800 mV, oxidation of CO is not occurred at the first measurement electrode 51 in the second internal cavity 40 even in the gas sensor 900. Therefore, by the pump current Ip2 at the voltage V1 of 800 mV, substantially all of the carbon monoxide generated by reduction (or, decomposition) of the carbon dioxide in the measurement-object gas can be considered to be oxidized at the second measurement electrode 44 in the third internal cavity 61. As the relative value (Ip2/Ip2_V1=800 mV) of the pump current Ip2 is close to 100% even at the lower voltage V1, the carbon monoxide is not likely to be oxidized in the second internal cavity 40, which is more preferable from the perspective of measurement accuracy.

As an evaluation index 2, a relative value (Ip2/Ip2_V1=800 mV) [%] of the pump current Ip2 when the voltage V1 was at 300 mV is shown in Table 1. It is considered that the effect of not oxidizing CO in the second internal cavity 40 becomes higher as the relative value (Ip2/Ip2_V1=800 mV) of the pump current Ip2 of the evaluation index 2 increases.

TABLE 1

| | | | | | Evaluation |
|---|---|---|---|---|---|
| | | | Ip1:Ip2 | Evaluation | index 2 |
| | | | (@V1 = | index 1 | Relative value |
| Sample | D2 | D3 | 300 mV) | V1 [mV] | of Ip2 [%] |
| Comparative | 2500 | 500 | 96:4 | 450.00 | 63.5 |
| Example 1 | | | | | |
| Example 1 | 100 | 500 | 87:12 | 480.00 | 60.8 |
| Example 2 | 500 | 500 | 49:51 | 530.00 | 66.0 |
| Example 3 | 3000 | 500 | 23:77 | 620.00 | 83.6 |
| Example 4 | 6000 | 500 | 22:78 | 645.00 | 90.6 |
| Example 5 | 9000 | 500 | 8:92 | 660.00 | 93.4 |

As shown in FIG. 13, it was confirmed that in each of Examples 1 to 5, the pump current Ip1 maintained at the limiting current until the higher voltage V1 in comparison with Comparative Example 1. It was also confirmed that the voltage V1 of the evaluation index 1 in each of Examples 1 to 5 was at the higher value compared with Comparative Example 1.

As shown in FIG. 14, it was confirmed that in each of Examples 3 to 5, oxidation of CO was reduced at the first measurement electrode 51 in the second internal cavity 40 even at the lower voltage V1 in comparison with Comparative Example 1. It was also confirmed that the relative value of the pump current Ip2 of the evaluation index 2 in each of Examples 3 to 5 was at the higher value compared with Comparative Example 1. The relative value of the pump current Ip2 of the evaluation index 2 in Examples 1 to 2 is roughly comparable to that in Comparative Example 1, however, it is considered that since the voltage V1 can be increase in terms of the limiting current of the pump current Ip1 as described above, oxidation of CO can be reduced at the first measurement electrode 51 in the second internal cavity 40.

As described above, according to the present invention, carbon monoxide is not oxidized in the second internal cavity 40 while the water vapor concentration is accurately measured by the limiting current, and it is therefore possible to measure the water vapor concentration with high accuracy and measure the carbon dioxide concentration with high accuracy. Therefore, it is possible to provide a gas sensor that can accurately measure both of a water vapor concentration and a carbon dioxide concentration in a measurement-object gas.

### EXPLANATION OF REFERENCE SIGNS IN THE DRAWINGS

1: first substrate layer; 2: second substrate layer; 3: third substrate layer; 4: first solid electrolyte layer; 5, 5a, 5b, 5c: spacer layer; 6: second solid electrolyte layer; 10: gas inlet; 11: first diffusion-rate limiting path; 12: buffer space; 13: auxiliary diffusion-rate limiting path; 15, 215, 915: measurement-object gas flow cavity; 20: first internal cavity; 21: main pump cell; 22: inner main pump electrode; 22a: ceiling electrode portion (of the inner main pump electrode); 22b: bottom electrode portion (of the inner main pump electrode); 23: outer pump electrode; 24: variable power supply (of the main pump cell); 30, 230, 930: second diffusion-rate limiting path; 40, 240, 940: second internal cavity; 41: second measurement pump cell; 42: reference electrode; 43: reference gas introduction space; 44: second measurement electrode; 46: variable power supply (of the second measurement pump cell); 48: air introduction layer; 50: first measurement pump cell; 51: first measurement electrode; 51a: ceiling electrode portion (of the first measurement pump cell); 51b: bottom electrode portion (of the first measurement pump cell); 52: variable power supply (of the first measurement pump cell); 60, 260, 960: third diffusion-rate limiting path; 61, 261, 961: third internal cavity; 70: heater part; 71: heater electrode; 72: heater; 73 through hole; 74 heater insulating layer; 75: pressure relief vent; 76: heater lead; 80: oxygen-partial-pressure detection sensor cell for main pump control; 81: oxygen-partial-pressure detection sensor cell for first measurement pump control; 82: oxygen-partial-pressure detection sensor cell for second measurement pump control; 83: sensor cell; 90: control unit; 91: control part; 92: pump control part; 93: concentration calculating part; 100, 200, 900: gas sensor; 101, 201, 901: sensor element; and 102, 202, 902: base part.

What is claimed is:

1. A gas sensor for detecting water vapor and carbon dioxide in a measurement-object gas, the gas sensor comprising a sensor element and a control unit for controlling the sensor element, wherein
the sensor element comprises:
a base part in an elongated plate shape, including an oxygen-ion-conductive solid electrolyte layer;
a measurement-object gas flow cavity having a gas inlet that opens on a surface of the base part, a first internal cavity that communicates with the gas inlet via a first diffusion-rate limiting path, a second internal cavity that communicates with the first internal cavity via a second diffusion-rate limiting path, and a third internal cavity that communicates with the first internal cavity via a third diffusion-rate limiting path;
an oxygen pump cell including an intracavity oxygen pump electrode disposed in the first internal cavity of the measurement-object gas flow cavity, and an extracavity oxygen pump electrode disposed at a position different from the measurement-object gas flow cavity on the base part and corresponding to the intracavity oxygen pump electrode;
a first measurement pump cell including an intracavity first measurement electrode disposed in the second internal cavity of the measurement-object gas flow cavity, and an extracavity first measurement electrode disposed at a position different from the measurement-object gas flow cavity on the base part and corresponding to the intracavity first measurement electrode; and
a second measurement pump cell including an intracavity second measurement electrode disposed in the third internal cavity of the measurement-object gas flow cavity, and an extracavity second measurement electrode disposed at a position different from the measurement-object gas flow cavity on the base part and corresponding to the intracavity second measurement electrode; and
the control unit comprises:
a pump control part for controlling operation of the oxygen pump cell, the first measurement pump cell and the second measurement pump cell, and
a concentration calculating part for calculating a concentration of each of water vapor and carbon dioxide in a measurement-object gas, wherein
the pump control part applies a predetermined oxygen pump voltage between the intracavity oxygen pump electrode and the extracavity oxygen pump electrode of the oxygen pump cell to pump out oxygen from the first internal cavity and decompose the water vapor and the carbon dioxide in the measurement-object gas in the first internal cavity;
the pump control part applies a predetermined first measurement pump voltage between the intracavity first measurement electrode and the extracavity first measurement electrode of the first measurement pump cell to pump oxygen into the second internal cavity and oxidize, in the second internal cavity, hydrogen generated by decomposition of the water vapor in the first internal cavity and introduced into the second internal cavity; and
the pump control part applies a predetermined second measurement pump voltage between the intracavity second measurement electrode and the extracavity second measurement electrode of the second measurement pump cell to pump oxygen into the third internal cavity, and oxidize, in the third internal cavity, hydrogen generated by the decomposition of the water vapor in the first internal cavity and introduced into the third internal cavity, and, carbon monoxide generated by decomposition of the carbon dioxide in the first internal cavity and introduced into the third internal cavity; and
the concentration calculating part calculates a water vapor concentration in the measurement-object gas based on a current flowing through the first measurement pump cell; and
the concentration calculating part calculates a carbon dioxide concentration in the measurement-object gas based on the current flowing through the first measurement pump cell and a current flowing through the second measurement pump cell.

2. The gas sensor according to claim 1, wherein the second internal cavity and the third internal cavity are formed independently of each other.

3. The gas sensor according to claim 1, wherein the measurement-object gas is distributed at a predetermined ratio and introduced into the second internal cavity from the first internal cavity via the second diffusion-rate limiting path, and into the third internal cavity from the first internal cavity via the third diffusion-rate limiting path, respectively.

4. The gas sensor according to claim 1, wherein
the sensor element further comprises:
a reference gas chamber formed inside the base part, and being separated from the measurement-object gas flow cavity; and
a reference electrode disposed in the reference gas chamber; and
the pump control part applies the predetermined oxygen pump voltage between the intracavity oxygen pump electrode and the extracavity oxygen pump electrode of the oxygen pump cell based on a voltage between the intracavity oxygen pump electrode and the reference electrode to pump out the oxygen from the first internal cavity and decompose the water vapor and the carbon dioxide in the measurement-object gas in the first internal cavity;
the pump control part applies the predetermined first measurement pump voltage between the intracavity first measurement electrode and the extracavity first measurement electrode of the first measurement pump cell based on a voltage between the intracavity first measurement electrode and the reference electrode to pump the oxygen into the second internal cavity and oxidize, in the second internal cavity, the hydrogen generated by the decomposition of the water vapor in the first internal cavity and introduced into the second internal cavity; and
the pump control part applies the predetermined second measurement pump voltage between the intracavity second measurement electrode and the extracavity second measurement electrode of the second measurement pump cell based on a voltage between the intracavity second measurement electrode and the reference electrode to pump the oxygen into the third internal cavity, and oxidize, in the third internal cavity, the hydrogen generated by the decomposition of the water vapor in the first internal cavity and introduced into the third internal cavity, and the carbon monoxide generated by the decomposition of the carbon dioxide and introduced into the third internal cavity.

5. The gas sensor according to claim 4, wherein the voltage between the intracavity oxygen pump electrode and the reference electrode is controlled at 300 mV or more and 700 mV or less.

6. The gas sensor according to claim 1, wherein a current ratio of the current flowing through the first measurement pump cell to the current flowing through the second measurement pump cell is smaller than 7.25, when the measurement-object gas with the water vapor concentration of 10% and nitrogen as a remainder is introduced into the measurement-object gas flow cavity.

7. The gas sensor according to claim 1, wherein a diffusion resistance of the second diffusion-rate limiting path is larger than a diffusion resistance of the third diffusion-rate limiting path.

8. The gas sensor according to claim 1, wherein the concentration calculating part calculates the carbon dioxide concentration in the measurement-object gas based on the water vapor concentration calculated based on the current flowing through the first measurement pump cell, and the current flowing through the second measurement pump cell.

9. The gas sensor according to claim 1, wherein the concentration calculating part further calculates an oxygen concentration in the measurement-object gas based on a current flowing through the oxygen pump cell, the current flowing through the first measurement pump cell, and the current flowing through the second measurement pump cell.

10. The gas sensor according to claim 9, wherein the concentration calculating part calculates the oxygen concentration in the measurement-object gas based on the current flowing through the oxygen pump cell, the water vapor concentration calculated based on the current flowing through the first measurement pump cell, and the carbon dioxide concentration calculated based on the current flowing through the first measurement pump cell and the current flowing through the second measurement pump cell.

11. A control method of a gas sensor for detecting water vapor and carbon dioxide in a measurement-object gas, the gas sensor being the gas sensor according to claim 1, and
the control method comprising:
a water vapor concentration measuring step of calculating a water vapor concentration in the measurement-object gas based on a current flowing through the first measurement pump cell; and
a carbon dioxide concentration measuring step of calculating a carbon dioxide concentration in the measurement-object gas based on the current flowing through the first measurement pump cell and a current flowing through the second measurement pump cell.
